(19) United States
(12) Patent Application Publication
(10) Pub. No.: US 2025/0066778 A1
(43) Pub. Date: Feb. 27, 2025

(54) METHODS AND MATERIALS FOR TREATING HEART ATTACK

(71) Applicant: THE JOHNS HOPKINS UNIVERSITY, Baltimore, MD (US)

(72) Inventor: Emmanouil TAMPAKAKIS, Baltimore, MD (US)

(21) Appl. No.: 18/726,381

(22) PCT Filed: Dec. 14, 2022
(86) PCT No.: PCT/US2022/052772

§ 371 Date: Jul. 2, 2024

Related U.S. Application Data
(60) Provisional application No. 63/296,028, filed on Jan. 3, 2022.
(60) Provisional application No. 63/322,049, filed on Mar. 21, 2022.

(51) Int. Cl. C12N 15/113; A61K 31/04; A61K 31/616; A61K 45/06; A61P 9/04; A61P 9/10

(52) U.S. Cl. CPC C12N 15/113; A61K 31/04; A61K 31/616; A61K 45/06; A61P 9/04; A61P 9/10; C12N 2310/14; C12N 2310/531

(57) ABSTRACT

This document relates to methods and materials for treating a mammal (e.g., a human) having, or at risk of having, a heart attack (also called a myocardial infarction). For example, one or more inhibitors of a period circadian protein (PER) polypeptide can be administered to a mammal (e.g., a human) having, or at risk of having, a heart attack to treat the mammal.

### CROSS-REFERENCE TO RELATED APPLICATIONS

This application claims the benefit of U.S. Patent Application Ser. No. 63/296,028, filed on Jan. 3, 2022, and U.S. Patent Application Ser. No. 63/322,049, filed on Mar. 21, 2022. The disclosures of the prior applications are considered part of (and are incorporated by reference in) the disclosure of this application.

### STATEMENT REGARDING FEDERAL FUNDING

This invention was made with government support under grant HL145135 awarded by the National Institutes of Health (NIH). The government has certain rights in the invention.

### TECHNICAL FIELD

This document relates to methods and materials for treating a mammal (e.g., a human) having, or at risk of having, heart disease (e.g., a heart attack (also called a myocardial infarction) or heart failure). For example, one or more inhibitors of a period circadian protein (PER) polypeptide can be administered to a mammal (e.g., a human) having, or at risk of having, heart disease (e.g., a heart attack or heart failure) to treat the mammal.

### BACKGROUND INFORMATION

Heart disease is the leading cause of death for men, women, and people of most racial and ethnic groups in the United States (Centers for Disease Control and Prevention. Underlying Cause of Death, 1999-2018. CDC WONDER Online Database. Atlanta, GA: Centers for Disease Control and Prevention; 2018). For example, every year in the United States, about 805,000 people have a heart attack (Fryar et al., Prevalence of uncontrolled risk factors for cardiovascular disease: United States, 1999-2010. NCHS data brief, no. 103. Hyattsville, MD: National Center for Health Statistics; 2012). Further, about 1 in 5 heart attacks is silent—the damage is done, but the person is not aware of it.

### SUMMARY

Postnatal cardiomyocyte maturation and withdrawal from cell cycle within the first week after birth accounts for the heart's limited ability to regenerate after injury (Tzahor et al., Science, 356:1035-1039 (2017)). Therefore, there is pressing interest to identify ways with which to regenerate the heart.

This document provides methods and materials for treating mammals (e.g., humans) having, or at risk of having, heart disease (e.g., a heart attack or heart failure). For example, this document provides methods for using inhibitors of a PER polypeptide for treating mammals (e.g., humans) having, or at risk of having, heart disease (e.g., a heart attack or heart failure). In some cases, one or more inhibitors of a PER polypeptide can be administered to a mammal (e.g., a human) having, or at risk of having, heart disease (e.g., a heart attack or heart failure) to treat the mammal. As demonstrated herein, downregulation of two clock gene homologs Period1 (Per1) and Period2 (Per2) can increase cardiomyocyte proliferation and heart size. As described herein, inhibitors of a PER polypeptide can increase cardiomyocyte proliferation and can be used for cardiac regeneration.

In general, one aspect of this document features methods for treating a mammal having a heart disease. The methods can include, or consist essentially of, administering an inhibitor of a PER polypeptide to a mammal having a heart disease. The mammal can be a human. The heart disease can be a heart attack or heart failure. The inhibitor of said PER polypeptide can inhibit one or more of a PER1 polypeptide, a PER2 polypeptide, and a PER3 polypeptide. The method can include identifying the mammal as having the heart disease prior to the administering. The inhibitor of the PER polypeptide can be an inhibitor of PER polypeptide activity. The inhibitor of the PER polypeptide can be an inhibitor of PER polypeptide expression. The inhibitor of the PER polypeptide expression can be a nucleic acid molecule designed to induce RNA interference of said PER polypeptide expression. The nucleic acid molecule designed to induce RNA interference of the PER polypeptide expression can be a short interfering RNA (siRNA). The nucleic acid molecule designed to induce RNA interference of the PER polypeptide expression can be a short hairpin RNA (shRNA). The method also can include administering an agent used to treat a heart attack to the mammal. The agent can be aspirin, thrombolytics, antiplatelet agents, anti-clotting agents, pain relievers, nitroglycerin, beta blockers, ace inhibitors, or statins. The method also can include subjecting the mammal to a therapy used to treat heart disease. The therapy can becoronary angioplasty, coronary stenting, or coronary artery bypass surgery.

In another aspect, this document features methods for increasing proliferation of a cardiomyocyte within a heart of a mammal having a heart disease. The methods can include, or consist essentially of, administering an inhibitor of a PER polypeptide to a mammal having a heart disease. The mammal can be a human. The heart disease can be a heart attack or heart failure. The inhibitor of said PER polypeptide can inhibit one or more of a PER1 polypeptide, a PER2 polypeptide, and a PER3 polypeptide. The method can include identifying the mammal as having the heart disease prior to the administering. The inhibitor of the PER polypeptide can be an inhibitor of PER polypeptide activity. The inhibitor of the PER polypeptide can be an inhibitor of PER polypeptide expression. The inhibitor of the PER polypeptide expression can be a nucleic acid molecule designed to induce RNA interference of said PER polypeptide expression. The nucleic acid molecule designed to induce RNA interference of the PER polypeptide expression can be a short interfering RNA (siRNA). The nucleic acid molecule designed to induce RNA interference of the PER polypeptide expression can be a short hairpin RNA (shRNA). The method also can include administering an agent used to treat a heart attack to the mammal. The agent can be aspirin, thrombolytics, antiplatelet agents, anti-clotting agents, pain relievers, nitroglycerin, beta blockers, ace inhibitors, or statins. The method also can include subjecting the mammal to a therapy used to treat heart disease. The therapy can becoronary angioplasty, coronary stenting, or coronary artery bypass surgery.

In another aspect, this document features methods for regenerating a cardiomyocyte within a heart of a mammal having a heart disease. The methods can include, or consist essentially of, administering an inhibitor of a PER polypeptide to a mammal having a heart disease. The mammal can be a human. The heart disease can be a heart attack or heart failure. The inhibitor of said PER polypeptide can inhibit one or more of a PER1 polypeptide, a PER2 polypeptide, and a PER3 polypeptide. The method can include identifying the mammal as having the heart disease prior to the administering. The inhibitor of the PER polypeptide can be an inhibitor of PER polypeptide activity. The inhibitor of the PER polypeptide can be an inhibitor of PER polypeptide expression. The inhibitor of the PER polypeptide expression can be a nucleic acid molecule designed to induce RNA interference of said PER polypeptide expression. The nucleic acid molecule designed to induce RNA interference of the PER polypeptide expression can be a short interfering RNA (siRNA). The nucleic acid molecule designed to induce RNA interference of the PER polypeptide expression can be a short hairpin RNA (shRNA). The method also can include administering an agent used to treat a heart attack to the mammal. The agent can be aspirin, thrombolytics, antiplatelet agents, anti-clotting agents, pain relievers, nitroglycerin, beta blockers, ace inhibitors, or statins. The method also can include subjecting the mammal to a therapy used to treat heart disease. The therapy can becoronary angioplasty, coronary stenting, or coronary artery bypass surgery.

In another aspect, this document features methods for increasing a number of cardiomyocytes within a heart of a mammal having a heart disease. The methods can include, or consist essentially of, administering an inhibitor of a PER polypeptide to a mammal having a heart disease. The mammal can be a human. The heart disease can be a heart attack or heart failure. The inhibitor of said PER polypeptide can inhibit one or more of a PER1 polypeptide, a PER2 polypeptide, and a PER3 polypeptide. The method can include identifying the mammal as having the heart disease prior to the administering. The inhibitor of the PER polypeptide can be an inhibitor of PER polypeptide activity. The inhibitor of the PER polypeptide can be an inhibitor of PER polypeptide expression. The inhibitor of the PER polypeptide expression can be a nucleic acid molecule designed to induce RNA interference of said PER polypeptide expression. The nucleic acid molecule designed to induce RNA interference of the PER polypeptide expression can be a short interfering RNA (siRNA). The nucleic acid molecule designed to induce RNA interference of the PER polypeptide expression can be a short hairpin RNA (shRNA). The method also can include administering an agent used to treat a heart attack to the mammal. The agent can be aspirin, thrombolytics, antiplatelet agents, anti-clotting agents, pain relievers, nitroglycerin, beta blockers, ace inhibitors, or statins. The method also can include subjecting the mammal to a therapy used to treat heart disease. The therapy can becoronary angioplasty, coronary stenting, or coronary artery bypass surgery.

In another aspect, this document features uses of a composition comprising an inhibitor of a PER polypeptide to treat a mammal having heart disease.

In another aspect, this document features inhibitors of a PER polypeptide for use as a medicament to treat a mammal having heart disease.

In another aspect, this document features inhibitors of a PER polypeptide for use in the treatment of a mammal having heart disease.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1L. Disruption of cardiac sympathetic innervation increases postnatal cardiomyocyte proliferation. FIG. 1A. Whole mount immunostaining of E18.5 mouse hearts for tyrosine hydroxylase (TH) showing inhibition of sympathetic innervation (SNi) in mutant (Sm22a-Cre; NGF fl/−) hearts vs controls (NGF fl/+). Scale bar: 250 μm. FIG. 1B. Whole mount immunofluorescent staining of P1 mouse atria and left ventricle (LV) showing persistently reduced epicardial sympathetic innervation. Scale bar: 100 μm. FIG. 1C. Immunostaining of P7 mouse hearts showing suppression of the epicardial to endocardial sympathetic innervation in the mutant heart atrium and left ventricle. Scale bar: 100 μm. FIG. 1D. Tyrosine hydroxylase protein expression is profoundly reduced in hearts with suppressed sympathetic innervation. FIG. 1E. Conscious mouse electrocardiogra recordings showing decreased average heart rates in mice with disrupted cardiac sympathetic innervation. FIG. 1F. Mutant mice developed increased heart rate standard deviation (SD) consistent with increased heart rate variability. FIG. 1G. Inhibited sympathetic innervation increased heart size, whereas body weight remained unchanged in mutant mice. FIG. 1H. Cardiomyocytes with inhibited sympathetic innervation were smaller. Scale bar: 10 μm. FIG. 1I. The total number of dissociated cardiomyocytes per high power field (hpf) was increased in hearts with inhibited SNs. FIG. 1J. Cardiomyocytes isolated from hearts with suppressed sympathetic innervation were more mononucleated and less binucleated. FIGS. 1K and 1L. Higher percentage of isolated P7 cardiomyocytes in hearts with suppressed sympathetic innervation were pH3+ and Edu+ as analyzed by flow cytometry. Student's t-test was used for two group analysis. Data are presented as mean±SEM. SNi: sympathetic neurons inhibition, TnT: Troponin T.

FIGS. 2A-2E. Inhibition of cardiac SNs results in upregulation of cell cycle genes and downregulation of Per1 and Per2 genes. FIG. 2A. Gene ontology (GO) analysis of RNA-sequencing data of P7 ventricles with disrupted sympathetic innervation compared to controls. Cell cycle, mitosis and nuclear division genes were upregulated (red), while circadian rhythm genes (green), calcium handling, cell size, muscle contraction, muscle action potential, heart rate and axonogenesis genes were downregulated. All hearts were isolated at the same time, ˜2 pm. FIG. 2B. Quantitative PCR analysis of cell cycle regulators showing increased expression of genes regulating S-phase and mitosis (M). FIG. 2C. Gene expression analysis of circadian genes, showing decrease expression of Per1 and Per2 circadian cycle regulators. FIG. 2D. Period 1 and Period 2 proteins were significantly reduced in hearts with suppressed sympathetic innervation. FIG. 2E. Calcium handling genes were decreased in hearts with disrupted sympathetic innervation. Student's t-test was used for two group analysis. Data are presented as mean±SEM. Only P values<0.1 are reported. SNi: sympathetic neurons inhibition.

FIGS. 3A-3G. Per1/Per2 DKO hearts have more proliferative neonatal cardiomyocytes. FIG. 3A. Per1/Per2 DKO mice developed increased heart size, whereas total body weight remained unchanged at P14. FIG. 3B. The size of individual Per1/Per2 DKO cardiomyocytes was decreased. FIG. 3C. The total number of cardiomyocytes in Per1/Per2 DKO hearts was increased. FIG. 3D. Per1/Per2 DKO myocytes were more mononucleated at P14. Scale bar: 20 μm. FIG. 3E. Immunofluorescent staining of P7 cardiac sections for pH3, showed more Per1/Per2 DKO cardiomyocytes entering mitosis. TnT: cardiac troponin T, scale bar: 25 μm. FIG. 3F. Immunofluorescent staining of P7 cardiac sections for Edu, showed more Per1/Per2 DKO cardiomyocytes in the S-phase. Scale bar: 25 μm. FIG. 3G. Gene expression analysis of the major cell cycle regulators showed increased expression of genes regulating mitosis. Student's t-test was used for two group analysis. Data are presented as mean±SEM. Only P values<0.1 are reported. SNi: sympathetic neurons inhibition, TnT: Troponin T.

FIGS. 4A-4C. Norepinephrine induces clock genes and suppress cell cycle genes in neonatal mouse cardiomyocytes. FIG. 4A. Norepinephrine concentration in heart with disrupted sympathetic innervation (SNi) is profoundly suppressed. FIG. 4B. Norepinephrine induces the expression of circadian genes in wild type neonatal mouse cardiomyocytes (NMCMs) after 48 hrs of treatment. FIG. 4C. Norepinephrine decreased the expression of mitosis regulating genes in wild type NMCMs. Norepinephrine failed to suppress mitosis regulating genes apart from Aurkb in cardiomyocytes isolated from Per1/Per2 DKO neonatal mouse hearts. Student's t-test was used for two group analysis. ANOVA test was used for multiple group comparisons. Data are presented as mean±SEM. Only P values<0.1 are reported.

FIGS. 5A-5E. In hearts with disrupted sympathetic cardiac innervation and in Per1/Per2 DKO hearts, suppression of Wee1 kinase activates the Cdk1/Cyclin B1 mitosis entry complex. FIG. 5A. Schematic representation of the different proteins linking the cell cycle with the circadian cycle. Wee1 is a kinase which phosphorylates and inactivates Cdk1 not allowing the Cdk1/CyclinB1 complex to induce entry into mitosis. Cdc25 is a phosphatase with the opposite effect. FIG. 5B. Gene expression analysis of factors known to link Per1/Per2 and cell cycle, showed that Wee1 is consistently decreased in both hearts with inhibited sympathetic innervation and Per1/Per2 DKO. Cdc25 is significantly increased in SNi hearts. FIGS. 5C and 5D. Western blot analysis confirmed decreased expression of Wee1 protein kinase and increased expression of Cdk1 and Cyclin B1 proteins in hearts with disrupted sympathetic innervation and Per1/Per2 DKO. Phosphorylated Tyr15 Cdk1 was decreased in both mouse models suggestive of increased Cdk1 activation. FIG. 5E. Norepinephrine treatment of wild type neonatal mouse cardiomyocytes induced Wee1 gene expression, however this effect was not observed in Per1/Per2 DKO cardiomyocytes. Student's t-test was used for two group analysis. ANOVA test was used for multiple group comparisons. Data are presented as mean±SEM. Only P values<0.1 are reported.

FIGS. 6A-6C. Per2 binds Wee1 and Cdk1 to likely regulate their expression. FIG. 6A. Chromatin immunoprecipitation-qPCR (ChIP-qPCR) analysis of P7 hearts using Per2 antibody showing increased enrichment of two regions within the Wee1 promoter in comparison to GAPDH control. FIG. 6B. Luciferase expression assay in human stem cell derived cardiomyocytes. Intact Wee1 promoter resulted in increased luciferase activity. Deletion of either Per2 binding sites reduced Luciferase activity, and the distal Per2 binding site had a more pronounced effect. NC=negative control. FIG. 6C. Schematic representation of a working model.

FIGS. 7A-7J. FIG. 7A. Breeding scheme to generate mice with disrupted cardiac sympathetic innervation. FIG. 7B. Fluorescent immunostaining of hearts isolated from Sm22a-Cre; Rosa-Tom/+ embryos at embryonic day 13.5 (E13.5). The base of the right (RV) and left (LV) ventricles are stained for tyrosine hydroxylase (TH) to demonstrate that Cre is expressed prior to the presence of any sympathetic innervation in the hearts. (scale bar: 40 μm). FIG. 7C. Whole mount immunostaining of Sm22a-Cre; NGF fl/− hearts for acetylcholine transporter (AChT) showing uninterrupted parasympathetic innervation. (scale bar: 100 μm). FIG. 7D. Mutant mice with inhibited sympathetic innervation develop greater standard deviation of successive RR interval differences (SDSD) consistent with increased heart rate variability. FIG. 7E. Representative conscious ECG recordings of mice with disrupted cardiac SNs and control mice over 12 hours showing increased heart variability in mutant mice. FIG. 7F. Flow cytometry analysis of dissociated P7 cardiomyocytes stained for the proliferation marker Ki67 showed a higher percentage of proliferating myocytes in hearts with inhibited sympathetic innervation. FIG. 7G. Negative control for Ki67 flow cytometry. FIGS. 7H-7I. Negative controls for pH3 and Edu flow cytometry. FIG. 7J. Cell cycle analysis of isolated P7 mouse cardiomyocytes. A higher percentage of single cardiomyocytes was observed in S and M phase, while less cardiomyocytes were in G0/G1 phase. Student's t-test was used for two group analysis. Data are presented as mean±SEM. SNi: sympathetic neurons inhibition.

FIGS. 8A-8C. FIG. 8A. Gene set enrichment analysis (GSEA) of RNA-sequencing data from P7 ventricles with disrupted sympathetic innervation compared to controls. Cell cycle and DNA replication genes were upregulated, while circadian rhythm, cardiomyopathy and muscle contraction genes were downregulated. FIG. 8B. Cytokinesis related genes were upregulated in hearts with inhibited sympathetic innervation. FIG. 8C. Cell cycle inhibitors were not differentially expressed in control hearts vs mutant hearts with disrupted SNs. Student's t-test was used for two group analysis. Data are presented as mean±SEM. Only P values<0.1 are reported. SNi: sympathetic neurons inhibition.

FIGS. 9A-9D. FIG. 9A. Sympathetic innervation of hearts without NGF expression (Mesp1-Cre; NGF fl/−) was mildly decreased. (scale bar: 200 μm). FIG. 9B. No difference in heart size (P14) in controls vs mice without cardiac NGF expression. FIGS. 9C-9D. There was no significant difference in cell cycle regulators and in circadian gene expression in hearts with suppressed NGF expression. Student's t-test was used for two group analysis. Data are presented as mean±SEM. Only P values<0.1 are reported.

FIG. 10. Per1 and Per2 genes remain persistently down-regulated in hearts with disrupted sympathetic innervation at P14. (All hearts were isolated at the same time, ˜10 am). Genes with an average log-fold change of at least 0.5 and a P-value<0.05 are reported.

FIGS. 11A-11F. The gene expression of the main circadian cycle regulators in P14 mouse hearts at prespecified 4-hour intervals. Circadian oscillations for Per1, Per2 and Clock were suppressed in SNi hearts.

FIGS. 12A-12F. FIG. 112A. Whole mount immunostaining of neonatal Per1/Per2 DKO hearts showed normal sympathetic innervation. (scale bar: 100 μm). FIG. 12B. A higher percentage of proliferating cardiomyocytes (Ki67+) was detected in Per1/Per2 DKO P7 hearts. (scale bar: 25 μm). FIG. 12C. Cell cycle analysis showed more Per1/Per2 DKO cardiomyocytes in S and M phase, while less cells were in G0/G1 phase. FIG. 12D. Calcium handling genes were decreased in Per1/Per2 DKO P7 hearts. FIG. 11E. No difference in the expression of cycle inhibitor genes in Per1/Per2 DKO P7 hearts. FIG. 12F. Cytokinesis-related genes were increased in Per1/Per2 DKO hearts. Student's t-test was used for two group analysis. Data are presented as mean±SEM. Only P values<0.1 are reported. TnT: cardiac troponin T.

FIG. 13. The expression of various clock genes in cultured NMCMs showed minimal variation from the mean after 2 hrs of treatment with 50% horse serum, suggesting that 50% horse serum can reset circadian gene expression in NMCMs.

FIGS. 14A-14B. FIG. 14A. Western blot analysis of activated phospho-ATM (Ser1982) protein in both hearts with disrupted sympathetic innervation and Per1/Per2 DKO showed no difference. FIG. 14B. Western blot analysis confirmed increased expression of Cdk2 protein in hearts with disrupted sympathetic innervation and Per1/Per2 DKO. Phosphorylated Tyr15 Cdk2 was decreased in both mouse models suggestive of increased Cdk2 activation. Data are presented as mean±SEM. Only P values<0.1 are reported. SNi: sympathetic neurons inhibition.

FIGS. 15A-15J. Seahorse XF96 measurements of oxygen consumption rate (OCR) in P14-P18 postnatal cardiomyocytes. FIG. 15A. A representative measurement of OCR in P14-P18 mouse from SNi vs controls. FIGS. 15B-15C. Basal OCR and ATP synthesis were reduced in SNi cardiomyocytes. FIGS. 15D-15E. There was no significant difference in max OCR and glycolysis (ECAR to OCR ratio) in these CMs. FIG. 15F. A representative measurement of OCR in P14-P18 mouse cardiomyocytes from Per1/Per2 DKO vs controls. FIGS. 15G-15J. There was no significant difference in OCR in Per1/Per2 DKO compared to controls.

FIG. 16. ChIP-qPCR analysis of P7 hearts using Per2 antibody showing enrichment of two intronic regions within the Cdk1 gene in comparison to GAPDH control.

### DETAILED DESCRIPTION

This document provides methods and materials for treating mammals (e.g., humans) having, or at risk of having, heart disease (e.g., a heart attack or heart failure). For example, this document provides methods for using inhibitors of a PER polypeptide. In some cases, one or more (e.g., one, two, three, four, or more) inhibitors of a PER polypeptide can be administered to a mammal (e.g., a human) having, or at risk of having, heart disease (e.g., a heart attack or heart failure) to treat the mammal. For example, one or more (e.g., one, two, three, four, or more) inhibitors of a PER polypeptide can be administered to a mammal (e.g., a human) having, or at risk of having, heart disease (e.g., a heart attack or heart failure) to regenerate cardiomyocytes within the heart of the mammal. In some cases, one or more (e.g., one, two, three, four, or more) inhibitors of a PER polypeptide can be administered to a mammal (e.g., a human) after has had a heart attack or heart failure to regenerate cardiomyocytes within the heart of the mammal.

In some cases, one or more (e.g., one, two, three, four, or more) inhibitors of a PER polypeptide can be used to reduce the severity of one or more symptoms of heart disease (e.g., a heart attack or heart failure). For example, one or more inhibitors of a PER polypeptide can be administered to a mammal (e.g., a human) in need thereof (e.g., a human having, or at risk of having, heart disease such as a heart attack or heart failure) to reduce the severity of one or more symptoms of the heart disease. For example, one or more inhibitors of a PER polypeptide can be administered to a mammal (e.g., a human) in need thereof (e.g., a human having, or at risk of having, heart disease such as a heart attack or heart failure) to reduce the severity of one or more symptoms of the heart disease. Examples of symptoms of heart disease include, without limitation, shortness of breath, chest pressure, tightness, pain, squeezing or aching in the chest or arms that may spread to your neck, jaw, or back, nausea, indigestion, heartburn, abdominal pain, cold sweat, fatigue, lightheadedness, sudden dizziness, palpitations, syncope, and swelling of the legs or the abdomen. In some cases, the methods and materials described herein can be effective to reduce the severity of one or more symptoms of heart disease in a mammal having, or at risk of having, heart disease (e.g., a heart attack or heart failure) by, for example, 10, 20, 30, 40, 50, 60, 70, 80, 90, 95, or more percent.

In some cases, one or more (e.g., one, two, three, four, or more) inhibitors of a PER polypeptide can be used to delay the onset of one or more symptoms of heart disease. For example, one or more inhibitors of a PER polypeptide can be administered to a mammal (e.g., a human) in need thereof (e.g., a human having, or at risk of having, heart disease such as a heart attack or heart failure) to delay the onset of one or more symptoms of the heart disease. For example, one or more inhibitors of a PER polypeptide can be administered to a mammal (e.g., a human) in need thereof (e.g., a human having, or at risk of having, heart disease such as a heart attack or heart failure) to delay the onset of one or more symptoms of the heart disease by from about 6 months to about 4 years (e.g., from about 6 months to about 3 years, from about 6 months to about 2 years, from about 6 months to about 1 years, from about 1 year to about 4 years, from about 2 years to about 4 years, from about 3 years to about 4 years, from about 1 year to about 2 years, from about 1.5 years to about 2.5 years, from about 2 years to about 3 years, or from about 2.5 years to about 3.5 years) or more. In some cases, one or more inhibitors of a PER polypeptide can be administered to a mammal (e.g., a human) in need thereof (e.g., a human having, or at risk of having, heart disease such as a heart attack or heart failure) to delay the onset of one or more symptoms of the heart disease by 6-12 months. In some cases, one or more inhibitors of a PER polypeptide can be administered to a mammal (e.g., a human) in need thereof (e.g., a human having, or at risk of having, heart disease such as a heart attack or heart failure) to delay the onset of one or more symptoms of the heart disease by 3-4 years.

In some cases, one or more (e.g., one, two, three, four, or more) inhibitors of a PER polypeptide can be used to reduce the severity of one or more complications associated with heart disease (e.g., a heart attack or heart failure). For example, one or more inhibitors of a PER polypeptide can be administered to a mammal (e.g., a human) in need thereof (e.g., a human having, or at risk of having, heart disease such as a heart attack or heart failure) to reduce the severity of one or more complications associated with the heart disease. Complications associated with heart disease can include, without limitation, abnormal heart rhythms (arrhythmias), sudden cardiac arrest, reduced heart function with subsequent symptoms of fatigue, shortness of breath, swelling, syncope, and lightheadedness. In some cases, the methods and materials described herein can be effective to reduce the severity of one or more complications associated with heart disease in a mammal having, or at risk of having, heart disease (e.g., a heart attack or heart failure) by, for example, 10, 20, 30, 40, 50, 60, 70, 80, 90, 95, or more percent.

In some cases, one or more (e.g., one, two, three, four, or more) inhibitors of a PER polypeptide can be used to delay the onset of one or more complications of heart disease (e.g., a heart attack or heart failure). For example, one or more inhibitors of a PER polypeptide can be administered to a mammal (e.g., a human) in need thereof (e.g., a human having, or at risk of having, heart disease such as a heart attack or heart failure) to delay the onset of one or more complications of the heart disease. For example, one or more inhibitors of a PER polypeptide can be administered to a mammal (e.g., a human) in need thereof (e.g., a human having, or at risk of having, heart disease such as a heart attack or heart failure) to delay the onset of one or more complications of the heart disease by from about 6 months to about 4 years (e.g., from about 6 months to about 3 years, from about 6 months to about 2 years, from about 6 months to about 1 years, from about 1 year to about 4 years, from about 2 years to about 4 years, from about 3 years to about 4 years, from about 1 year to about 2 years, from about 1.5 years to about 2.5 years, from about 2 years to about 3 years, or from about 2.5 years to about 3.5 years) or more. In some cases, one or more inhibitors of a PER polypeptide can be administered to a mammal (e.g., a human) in need thereof (e.g., a human having, or at risk of having, heart disease such as a heart attack or heart failure) to delay the onset of one or more complications of the heart disease by 6-12 months. In some cases, one or more inhibitors of a PER polypeptide can be administered to a mammal (e.g., a human) in need thereof (e.g., a human having, or at risk of having, heart disease such as a heart attack or heart failure) to delay the onset of one or more complications of the heart disease by 3-4 years.

In some cases, one or more (e.g., one, two, three, four, or more) inhibitors of a PER polypeptide can be used to extend the life expectancy of a mammal having, or at risk of having, heart disease (e.g., a heart attack or heart failure). For example, one or more inhibitors of a PER polypeptide can be administered to a mammal (e.g., a human) in need thereof (e.g., a human having, or at risk of having, heart disease such as a heart attack or heart failure) to extend the life expectancy of the mammal. For example, one or more inhibitors of a PER polypeptide can be administered to a mammal (e.g., a human) in need thereof (e.g., a human having, or at risk of having, heart disease such as a heart attack or heart failure) to extend the life expectancy of the mammal by from about 1 year to about 5 years (e.g., from about 1 year to about 4 years, from about 1 year to about 3 years, from about 1 year to about 2 years, from about 2 years to about 5 years, from about 3 years to about 5 years, from about 4 years to about 5 years, from about 2 years to about 4 years, from about 2 years to about 3 years, or from about 3 years to about 4 years) or longer.

In some cases, one or more (e.g., one, two, three, four, or more) inhibitors of a PER polypeptide can be used to increase the amount of cardiomyocyte proliferation in the heart of a mammal having, or at risk of having, heart disease (e.g., a heart attack or heart failure). For example, one or more inhibitors of a PER polypeptide can be administered to a mammal (e.g., a human) in need thereof (e.g., a human having, or at risk of having, heart disease such as a heart attack or heart failure) to increase number of proliferating cardiomyocytes in the heart of the mammal. In some cases, the methods and materials described herein can be effective to increase number of proliferating cardiomyocytes in the heart of a mammal having, or at risk of having, heart disease (e.g., a heart attack or heart failure) by, for example, 10, 20, 30, 40, 50, 60, 70, 80, 90, 95, or more percent. For example, one or more inhibitors of a PER polypeptide can be administered to a mammal (e.g., a human) in need thereof (e.g., a human having, or at risk of having, heart disease such as a heart attack or heart failure) to increase the rate of proliferation of cardiomyocytes in the heart of the mammal. In some cases, the methods and materials described herein can be effective to increase the rate of proliferation of cardiomyocytes in the heart of a mammal having, or at risk of having, heart disease (e.g., a heart attack or heart failure) by, for example, from about 5 percent to about 10 percent.

In some cases, one or more (e.g., one, two, three, four, or more) inhibitors of a PER polypeptide can be used to regenerate cardiomyocytes within a heart. For example, one or more inhibitors of a PER polypeptide can be administered to a mammal (e.g., a human) in need thereof (e.g., a human having, or at risk of having, heart disease such as a heart attack or heart failure) to increase the number of cardiomyocytes present within the heart of a mammal. In some cases, the methods and materials described herein can be effective to increase the number of cardiomyocytes within the heart of a mammal having, or at risk of having, heart disease (e.g., a heart attack or heart failure) by, for example, about 10 percent.

In some cases, one or more (e.g., one, two, three, four, or more) inhibitors of a PER polypeptide can be used to regulate expression of one or more cell cycle genes in the cardiomyocytes within a mammal having, or at risk of having, heart disease (e.g., a heart attack or heart failure). For example, one or more inhibitors of a PER polypeptide can be administered to a mammal (e.g., a human) in need thereof (e.g., a human having, or at risk of having, heart disease such as a heart attack or heart failure) to increase expression of a Cdk1polypeptide (e.g., an active (unphosphorylated) Cdk1 polypeptide), a CyclinB1 polypeptide, or a combination thereof. For example, one or more inhibitors of a PER polypeptide can be administered to a mammal (e.g., a human) in need thereof (e.g., a human having, or at risk of having, heart disease such as a heart attack or heart failure) to decrease expression of a Wee1polypeptide.

Any appropriate mammal having, or at risk of having, heart disease (e.g., a heart attack or heart failure) can be treated as described herein (e.g., by administering one or more inhibitors of a PER polypeptide). Examples of mammals that can have heart disease (e.g., a heart attack or heart failure) and can be treated as described herein include, without limitation, humans, non-human primates such as monkeys, dogs, cats, horses, cows, pigs, sheep, mice, and rats. In some cases, a human can be treated as described herein. For example, a human having high blood pressure and having, or at risk of having, heart disease (e.g., a heart attack or heart failure) can be treated as described herein. For example, a human having high blood cholesterol levels and having, or at risk of having, heart disease (e.g., a heart attack or heart failure) can be treated as described herein. For example, a human having high triglyceride levels and having, or at risk of having, heart disease (e.g., a heart attack or heart failure) can be treated as described herein. For example, an obese human having, or at risk of having, heart disease (e.g., a heart attack or heart failure) can be treated as described herein.

In some cases, the methods described herein can include identifying a mammal (e.g., a human) as having, or at risk of having, heart disease (e.g., a heart attack or heart failure). Any appropriate method can be used to identify a mammal as having, or at risk of having, heart disease (e.g., a heart attack or heart failure). For example, electrocardiogram (ECG), blood tests (e.g., to check for the presence of enzymes associate with heart attack), chest X-rays, echocardiograms, coronary catheterization (angiograms), cardiac computed tomography/angiography (CTA), and/or cardiac magnetic resonance imaging (MRI) can be used to identify mammals (e.g., humans) having heart disease (e.g., a heart attack or heart failure). For example, high blood cholesterol, high blood pressure, diabetes, family history of heart disease, smoking, and/or obesity can be used to identify mammals (e.g., humans) at risk of having heart disease (e.g., a heart attack or heart failure).

A mammal (e.g., a human) having, or at risk of having, heart disease (e.g., a heart attack or heart failure) can be administered or instructed to self-administer any one or more (e.g., one, two, three, four, or more) inhibitors of a PER polypeptide. An inhibitor of a PER polypeptide can inhibit any appropriate PER polypeptide. Examples of PER polypeptides include, without limitation, PER1 polypeptides, PER2 polypeptides, and PER3 polypeptides. In some cases, PER polypeptides can be as set forth in National Center for Biotechnology Information (NCBI) accession no. 5183, accession no. 8864, and accession no. 8863.

An inhibitor of a PER polypeptide can be an inhibitor of PER polypeptide activity or an inhibitor of PER polypeptide expression. Examples of compounds that can reduce or eliminate PER polypeptide activity include, without limitation, small molecules that target (e.g., target and bind) to a PER polypeptide. Examples of inhibitors of PER polypeptide activity include, without limitation, PF670462 (Tocris, 3316) and PF4800567 (Tocris, 4281/10). When a compound that can reduce or eliminate PER polypeptide activity is a small molecule that targets (e.g., targets and binds) to a PER polypeptide, the small molecule can be in the form of a salt (e.g., a pharmaceutically acceptable salt).

Examples of compounds that can reduce or eliminate PER polypeptide expression include, without limitation, nucleic acid molecules designed to induce RNA interference of PER polypeptide expression (e.g., a short interfering RNA (siRNA) molecule or a short hairpin RNA (shRNA) molecule), antisense molecules, and miRNAs. Examples of compounds that can reduce or eliminate PER polypeptide expression include, without limitation, Per1 siRNA (Dharmacon, J-011350-05-0005), Per 2 siRNA (Dharmacon, J-012977-05-0005), Per1 shRNA (Sigma-Millipore, TRCN0000074184), and Per2 shRNA (Sigma MIllipore, TRCN0000018539).

Additional nucleic acid molecules designed to induce RNAi against PER polypeptide expression can be designed based on any appropriate nucleic acid encoding a PER polypeptide sequence. Examples of nucleic acids encoding a PER polypeptide sequence include, without limitation, those set forth in NCBI accession no. NM_002616.3 and accession no. NM_022817.3.

In some cases, an inhibitor of a PER polypeptide can be as described elsewhere (see, e.g., Sundaram et al., Sci. Rep., 9(1): 13743 (2019); Miller et al., J. Mol. Biol., 432(12): 3498-3514 (2020); He et al., Curr. Drug Metab., 17(5): 503-12 (2016); and Ruan et al., Nat. Rev. Drug Discov., 20(4): 287-307 (2021)).

In some cases, one or more inhibitors of a PER polypeptide can be formulated into a composition (e.g., a pharmaceutically acceptable composition) for administration to a mammal (e.g., a human) having, or at risk of having, heart disease (e.g., a heart attack or heart failure). For example, one or more inhibitors of a PER polypeptide can be formulated together with one or more pharmaceutically acceptable carriers (additives), excipients, and/or diluents. Examples of pharmaceutically acceptable carriers, excipients, and diluents that can be used in a composition described herein include, without limitation, cyclodextrins (e.g., beta-cyclodextrins such as KLEPTOSE®), dimethylsulfoxide (DMSO), sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline such as phosphate buffered saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, when a composition containing one or more inhibitors of a PER polypeptide is administered to a mammal (e.g., a human) having, or at risk of having, heart disease (e.g., a heart attack or heart failure), the composition can be designed for oral or parenteral (including, without limitation, a subcutaneous, intramuscular, intravenous, intradermal, intra-cerebral, intrathecal, or intraperitoneal injection) administration to the mammal. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

A composition containing one or more inhibitors of a PER polypeptide can be administered to a mammal (e.g., a human) having, or at risk of having, heart disease (e.g., a heart attack or heart failure) in any appropriate amount (e.g., any appropriate dose). An effective amount of a composition containing one or more inhibitors of a PER polypeptide can be any amount that can treat a mammal having, or at risk of having, heart disease (e.g., a heart attack or heart failure) as described herein without producing significant toxicity to the mammal. In cases where an inhibitor of a PER polypeptide is an inhibitor of PER polypeptide activity, an effective amount of one or more inhibitors of a PER polypeptide can be from about 10 milligrams per kilogram body weight (mg/kg) to about 30 mg/kg. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the heart attack in the mammal being treated may require an increase or decrease in the actual effective amount administered.

A composition containing one or more inhibitors of a PER polypeptide can be administered to a mammal (e.g., a human) having, or at risk of having, heart disease (e.g., a heart attack or heart failure) in any appropriate frequency. The frequency of administration can be any frequency that can treat a mammal having, or at risk of having, heart disease (e.g., a heart attack or heart failure) without producing significant toxicity to the mammal. For example, the frequency of administration can be from about once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more inhibitors of a PER polypeptide can be administered to a mammal (e.g., a human) having, or at risk of having, heart disease (e.g., a heart attack or heart failure) for any appropriate duration. An effective duration for administering or using a composition containing one or more inhibitors of a PER polypeptide can be any duration that can treat a mammal having, or at risk of having, heart disease (e.g., a heart attack or heart failure) without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human) having, or at risk of having, heart disease (e.g., a heart attack or heart failure) can include administering to the mammal one or more inhibitors of a PER polypeptide as the sole active ingredient to treat the mammal. For example, a composition containing one or more inhibitors of a PER polypeptide can include the one or more inhibitors of a PER polypeptide as the sole active ingredient in the composition that is effective to treat a mammal having, or at risk of having, heart disease (e.g., a heart attack or heart failure).

In some cases, methods for treating a mammal (e.g., a human) having, or at risk of having, heart disease (e.g., a heart attack or heart failure) as described herein (e.g., by administering one or more inhibitors of a PER polypeptide) also can include administering to the mammal one or more (e.g., one, two, three, four, five or more) additional agents and/or therapies used to treat heart disease (e.g., a heart attack or heart failure). For example, a combination therapy used to treat heart disease (e.g., a heart attack or heart failure) can include administering to the mammal (e.g., a human) one or more inhibitors of a PER polypeptide described herein and one or more (e.g., one, two, three, four, five or more) agents used to treat heart disease (e.g., a heart attack or heart failure). Examples of agents that can be administered to a mammal to treat heart disease (e.g., a heart attack or heart failure) include, without limitation, aspirin, thrombolytics, antiplatelet agents, anti-clotting agents, pain relievers, nitroglycerin, beta blockers, ace inhibitors, and statins any combinations thereof. In cases where one or more inhibitors of a PER polypeptide are used in combination with additional agents used to treat heart disease (e.g., a heart attack or heart failure), the one or more additional agents can be administered at the same time (e.g., in a single composition containing both one or more inhibitors of a PER polypeptide and the one or more additional agents) or independently. For example, one or more inhibitors of a PER polypeptide described herein can be administered first, and the one or more additional agents administered second, or vice versa.

In some cases, a combination therapy used to treat heart disease (e.g., a heart attack or heart failure) can include administering to the mammal (e.g., a human) one or more inhibitors of a PER polypeptide described herein and performing one or more (e.g., one, two, three, four, five or more) additional therapies used to treat heart disease (e.g., a heart attack or heart failure) on the mammal. Examples of therapies used to treat heart disease (e.g., a heart attack or heart failure) include, without limitation, coronary angioplasty, coronary stenting, and/or coronary artery bypass surgery. In cases where one or more inhibitors of a PER polypeptide described herein are used in combination with one or more additional therapies used to treat heart disease (e.g., a heart attack or heart failure), the one or more additional therapies can be performed at the same time or independently of the administration of one or more inhibitors of a PER polypeptide described herein. For example, one or more inhibitors of a PER polypeptide described herein can be administered before, during, or after the one or more additional therapies are performed.

The invention will be further described in the following examples, which do not limit the scope of the invention described in the claims.

### EXAMPLES

### Example 1: Heart Neurons Use Clock Genes to Control Myocyte Proliferation

This Example demonstrates that sympathetic neurons (SNs) can regulate Per1/Per2 oscillations in the heart, and that suppression Per1/Per2 can increase postnatal cardiomyocyte proliferation. For example, one or more inhibitors of a PER polypeptide (e.g., a PER1 polypeptide and/or a PER2 polypeptide) can increase cardiomyocyte proliferation and cardiomyocyte entry into mitosis.

### Results

### Sympathetic Innervation Inhibits Postnatal Cardiomyocyte Proliferation

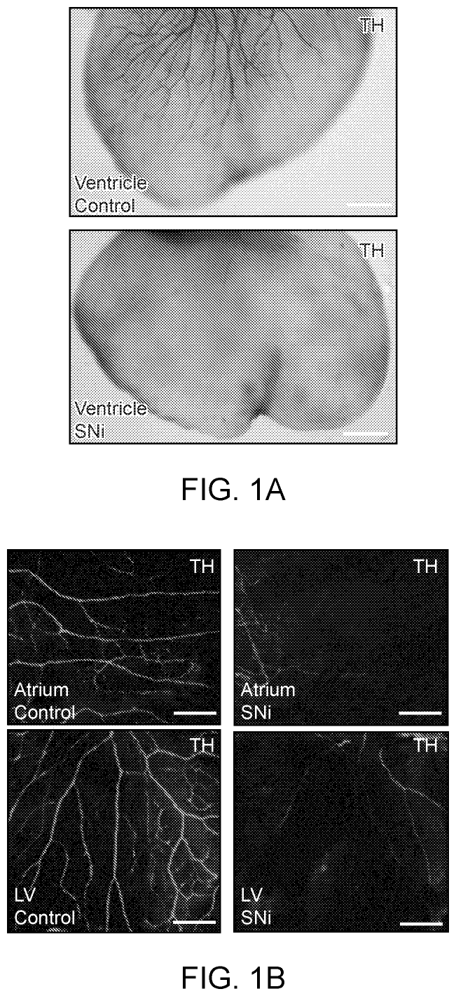

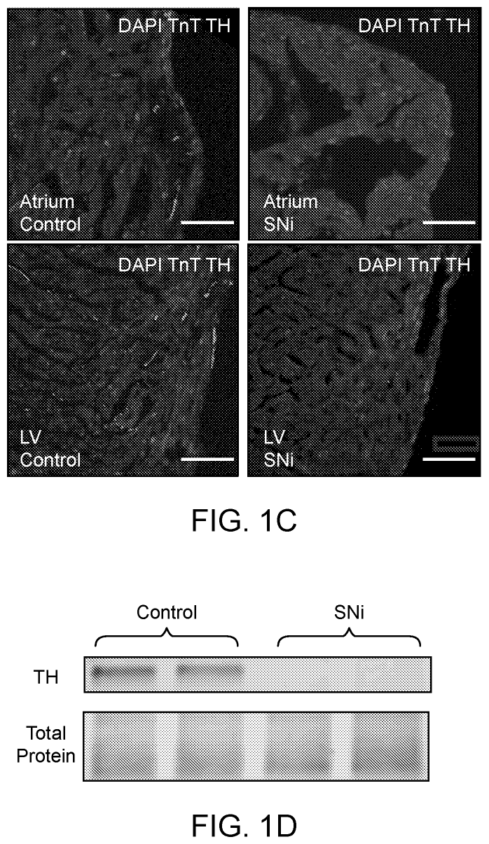

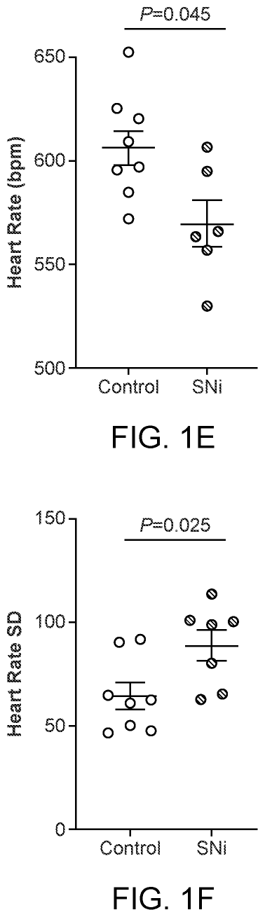

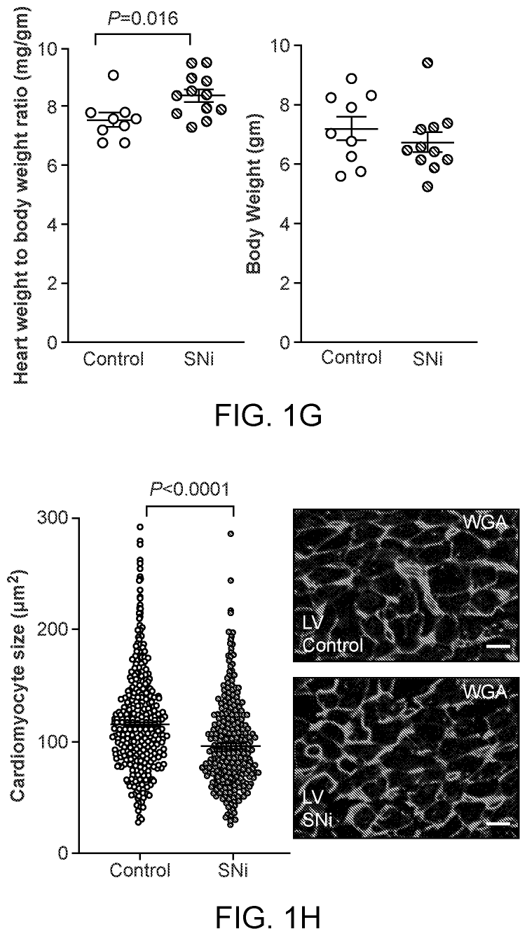

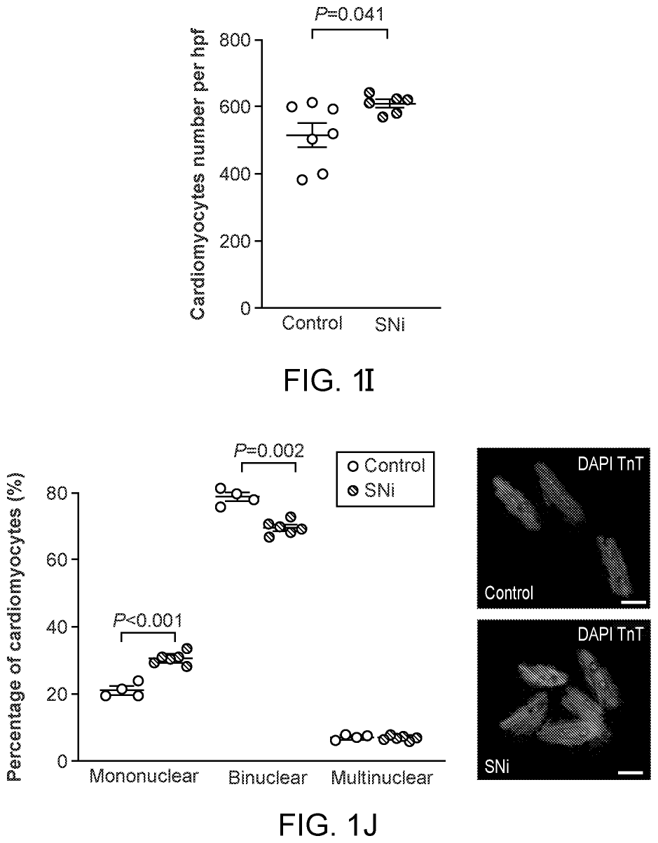

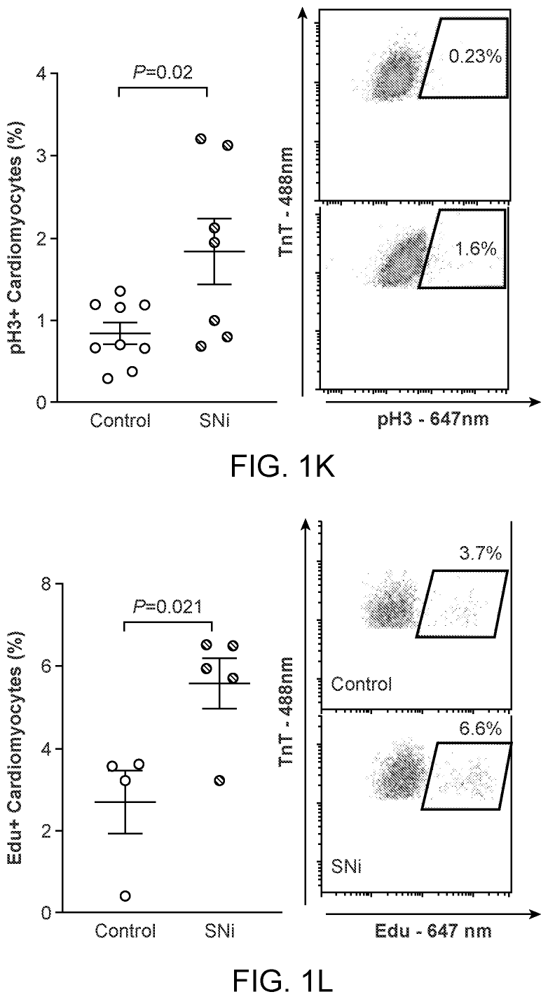

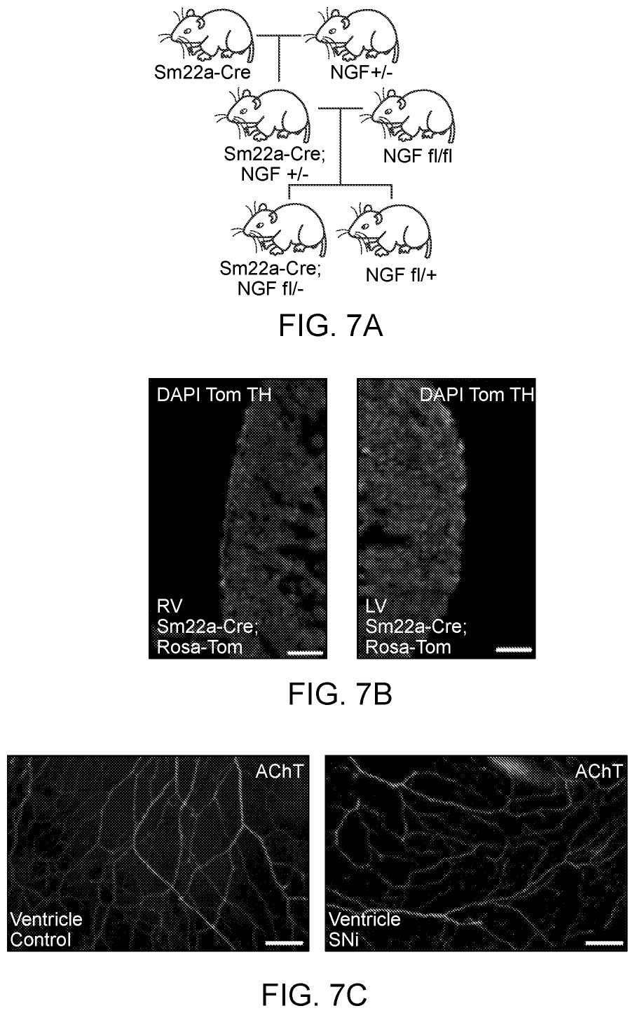

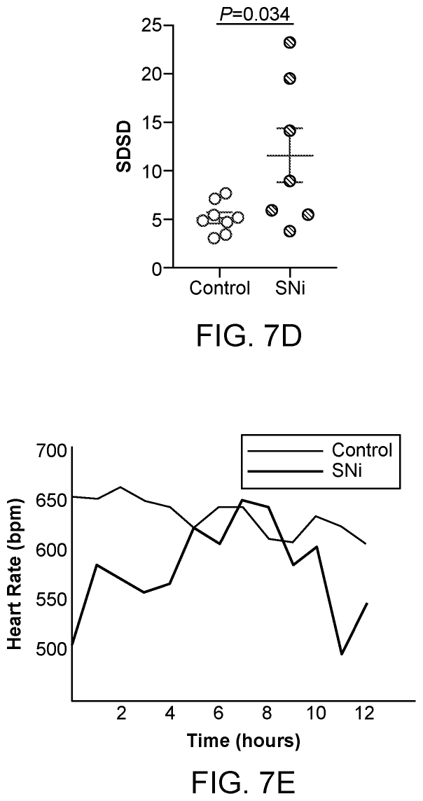

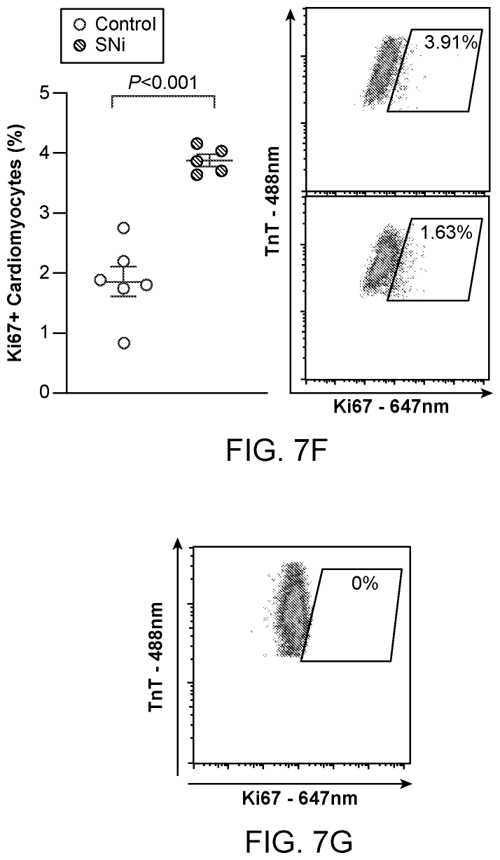

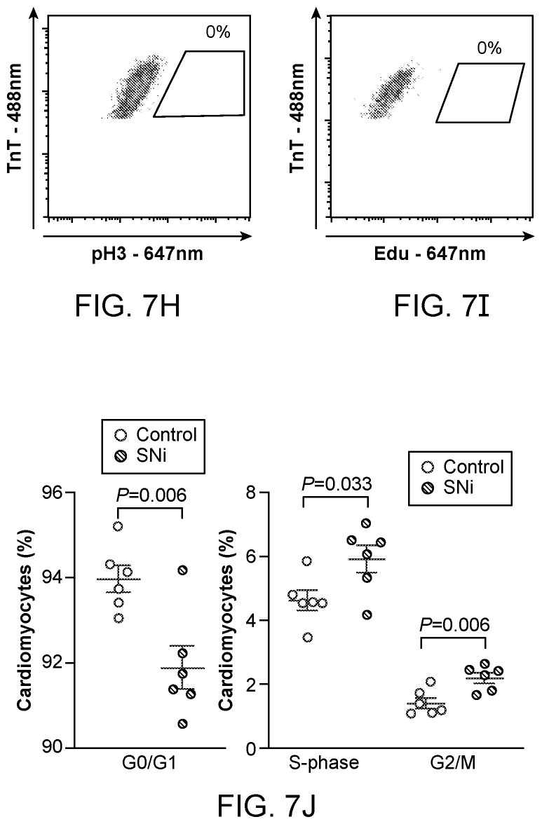

To investigate the role of SNs in postnatal heart development, and avoid the early lethality, NGF was conditionally deleted in smooth muscle cells (Sm22a-Cre; NGF fl/−) and SN innervation was monitored (FIG. 7A). It was first confirmed that Sm22a-Cre is expressed prior to cardiac innervation at E13.5 (FIG. 7B) and then examined the degree of sympathetic innervation by performing whole mount immunostaining of E18.5 and postnatal day (P) 1 mice for tyrosine hydroxylase (TH) a marker of SNs. As compared to control hearts, the mutant hearts exhibited a profound suppression of SN innervation (FIG. 1A-1B), whereas immunostaining for acetylcholine transporter (AChT) a marker of parasympathetic neurons showed uninterrupted parasympathetic innervation (FIG. 7C). Similarly, immunofluorescent staining of cardiac sections of P7 mice showed the inhibition of SNs and the disturbance of the epicardial to endocardial innervation pattern in both atria and ventricles (FIG. 1C). It was also confirmed that the total tyrosine hydroxylase protein expression in hearts with disrupted SNs was profoundly suppressed (FIG. 1D). To verify the physiologic effect of suppressed sympathetic innervation, conscious electrocardiogramgs of early adult mice were performed and it was confirmed that they had lower average heart rates and increased heart rate variability, indicating reduced sympathetic activity (FIG. 1E-1F, FIG. 7D-7E).

To examine if sympathetic innervation affects the hyperplastic to hypertrophic transition of postnatal cardiomyocytes, the heart growth was first analyzed. SN-deficient hearts (at P14) become larger in size (FIG. 1G). Next, the size and number of the resulting cardiomyocytes were quantified. Cardiomyocytes became smaller in size (FIG. 1H), whereas their total number was significantly increased in hearts lacking SNs (FIG. 1J). This suggests that SNs suppress cardiomyocyte hypertrophy and increase the number of cardiomyocytes in postnatal hearts. The number of nuclei per cardiomyocyte was analyzed and it was found that mutant hearts have a significantly higher percentage of mononuclear cardiomyocytes (FIG. 1J). To test whether the higher number of myocytes is a result of increased proliferation, immunofluorescent staining of dissociated postnatal cardiomyocytes was performed and cells were analyzed by flow cytometry. SN-deficient hearts displayed a higher percentage of myocytes positive for Ki67, a marker of proliferation expressed throughout the cell cycle (Ki67+, FIG. 7F-7G). Further analysis showed increased percentages of cardiomyocytes positive for phosphohistone H3 (pH3) and 5-ethynyl-2′-deoxyuridine (EdU) (FIG. 1K-1L, FIG. 7H-7I). Cell cycle analysis of isolated single cardiomyocytes from P7 SNi hearts was also performed and it was verified that a higher percentage of cells are in the S and mitosis phase, while a lower proportion are in G0/G1 phase (FIG. 7J). Together, these data indicate that in the absence of SNs, more cardiomyocytes undergo DNA replication and enter mitosis, resulting in an increase in cell number and percentage of mononuclear myocytes.

Disruption of sympathetic neurons upregulates cell cycle genes and downregulates clock genes in postnatal hearts

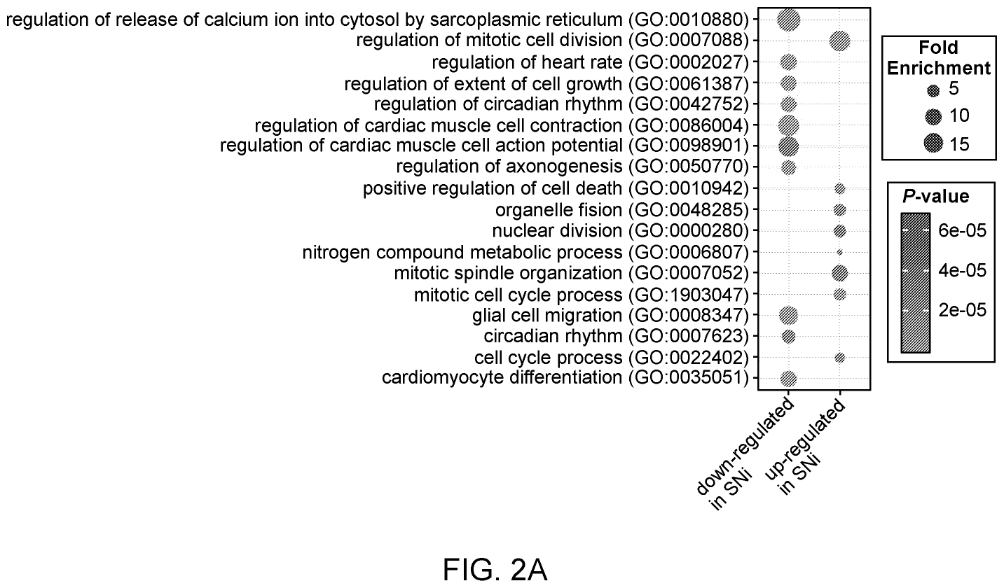

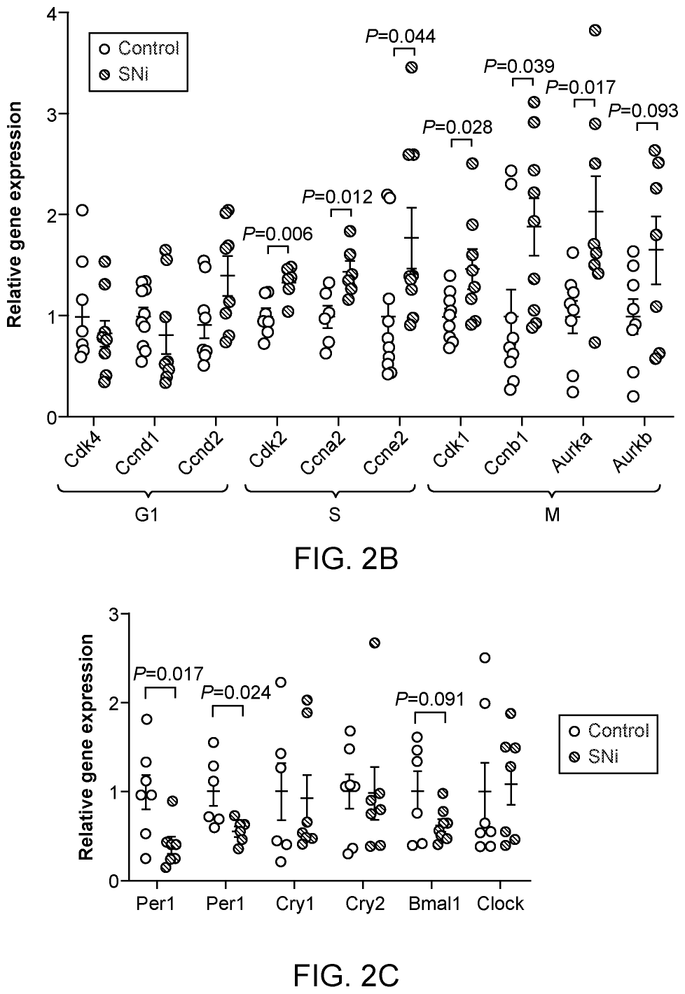

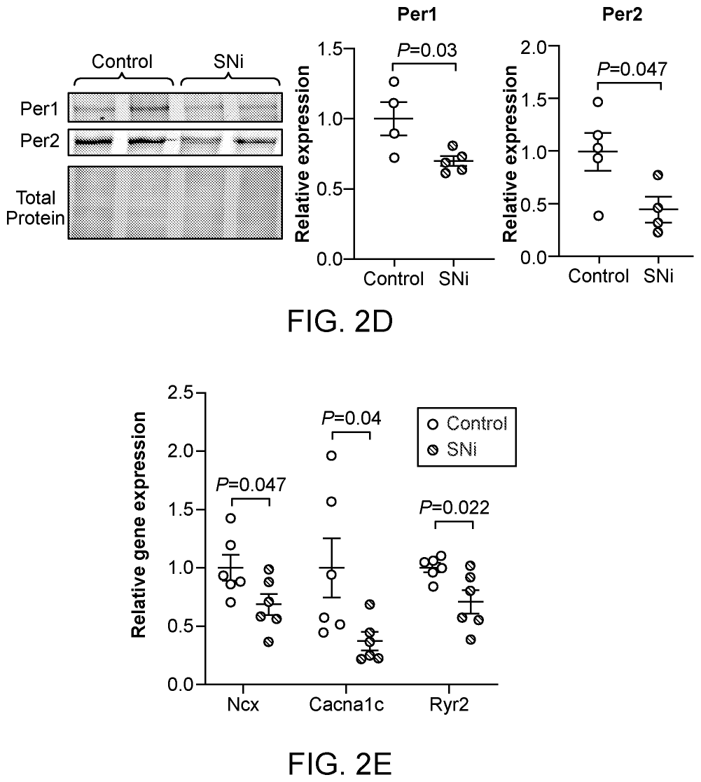

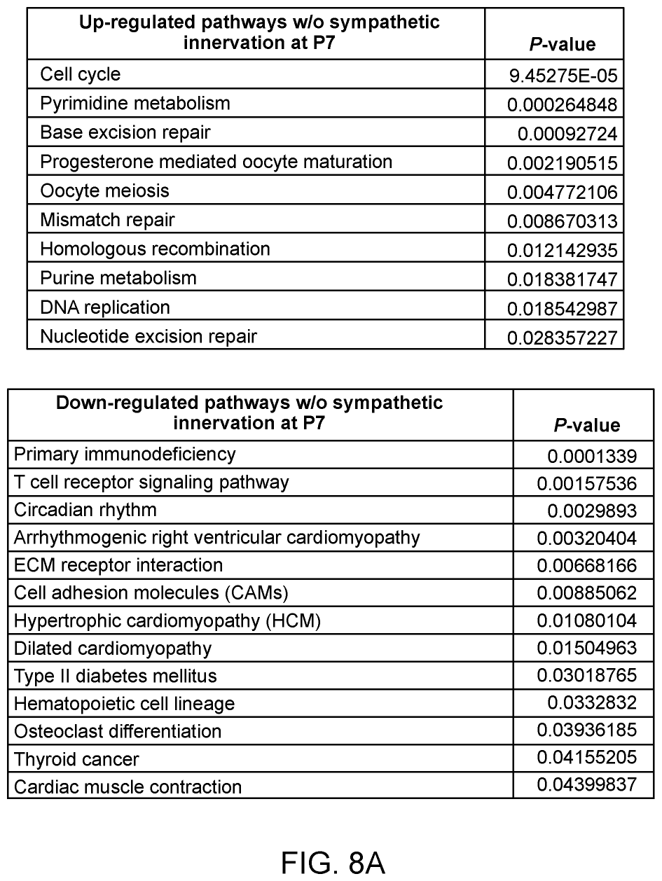

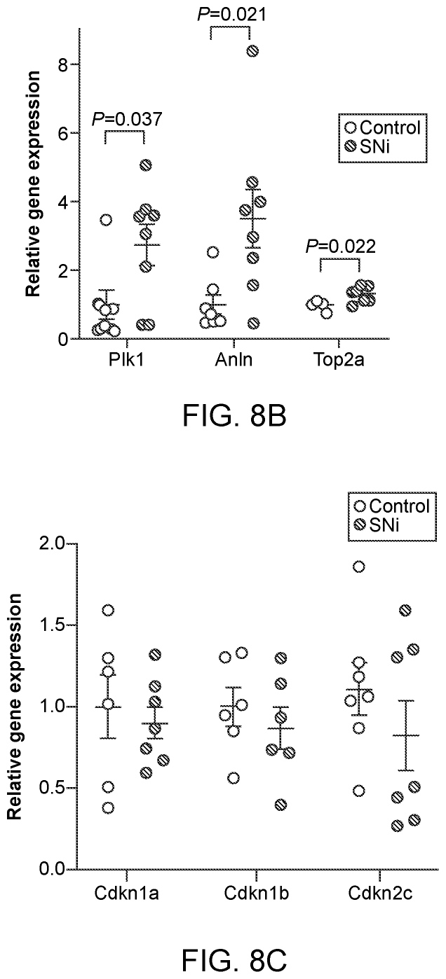

To gain mechanistic insights on the downstream pathways of cardiac SN signals, RNA-sequencing of postnatal hearts with suppressed SNs and littermate controls was performed. Gene ontology (GO) and gene set enrichment analyses (GSEA) showed the upregulation of cell cycle, mitosis, and DNA replication genes in hearts with suppressed SNs (FIG. 2A, FIG. 8A), supporting the observed phenotype. Genes regulating the circadian cycle were significantly downregulated (FIG. 2A, FIG. 8A). Furthermore, calcium handling, muscle contraction, and cardiomyopathy related genes were decreased in hearts with suppressed sympathetic innervation, implying their potential role in functional maturation of cardiomyocytes (FIG. 2A, FIG. 8A).

Based on the phenotypic and transcriptomic analysis, how SNs regulate cardiomyocyte proliferation was next analyzed. To determine which phase of the cell cycle was affected, the expression of the main cell cycle regulators was examined. While expression of G1 phase genes was unchanged, genes regulating the S-phase and mitosis were significantly increased in hearts with inhibited SNs (FIG. 2B). Accordingly, several cytokinesis related genes were also upregulated (FIG. 8B), but no significant difference was observed in the expression of cell cycle inhibitors (FIG. 8C). These data indicate that SNs control cardiomyocyte proliferation by suppressing S-phase and mitosis activators.

### NGF Does Not Affect Cardiomyocyte Proliferation

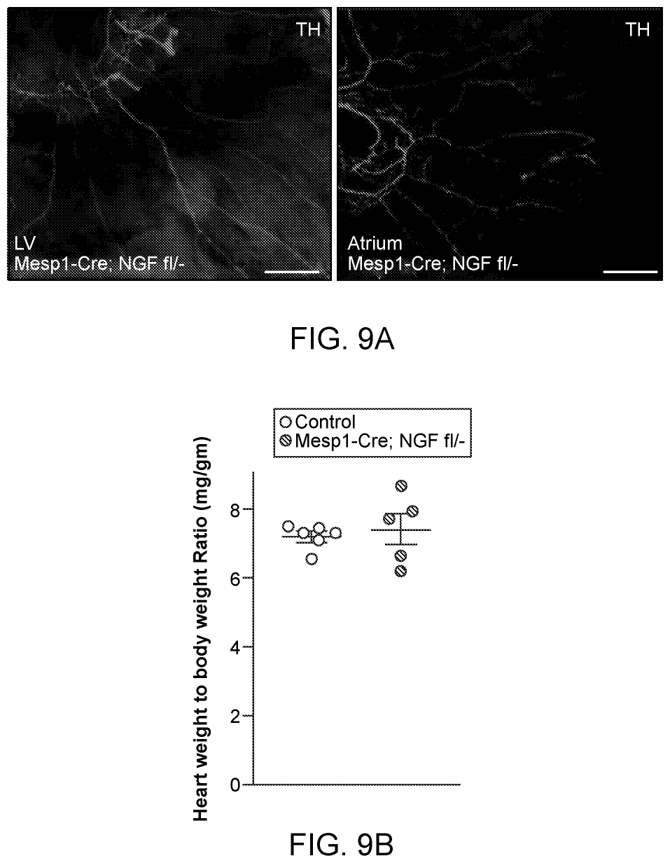

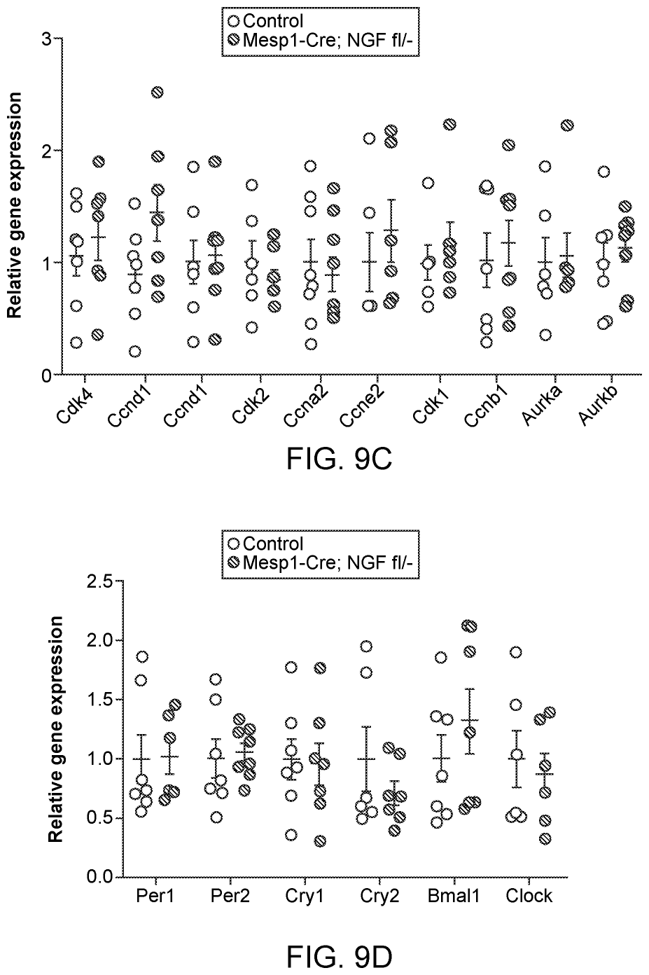

To exclude any confounding effect resulting from decreased NGF expression in the heart, NGF was deleted in Mesp1+ cells (giving rise to all cardiac lineages) using the Mesp1-Cre driver. The deletion reduced SN innervation mildly (FIG. 9A), which is likely due to the fact that a proportion of cardiac smooth muscle cells derives from neural crest cells (Wnt-1+) and not exclusively from Mesp1+ cells. Additionally, the heart size remained unchanged, and cell cycle as well as clock genes were not significantly affected by the deletion (FIG. 9B-9D). Similarly, ex vivo treatment of neonatal mouse cardiomyocytes (NMCMs) with NGF, did not affect cell cycle gene expression (FIG. 9E). This suggests that the observed increased cardiomyocyte proliferation was not caused by reduced NGF expression.

### Period1 Period2 Deletion Increases Cardiomyocyte Proliferation

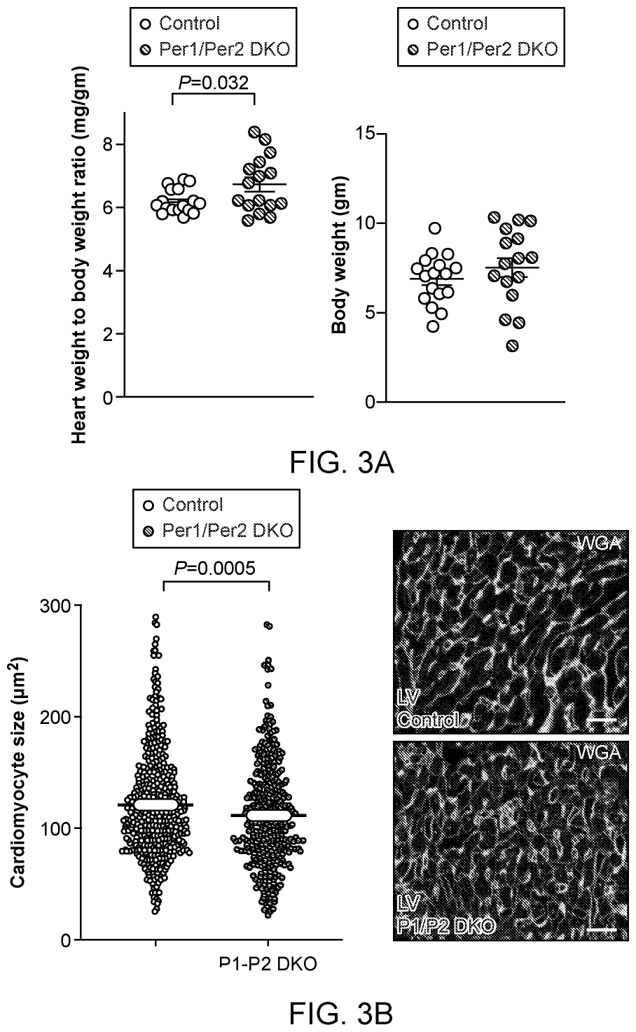

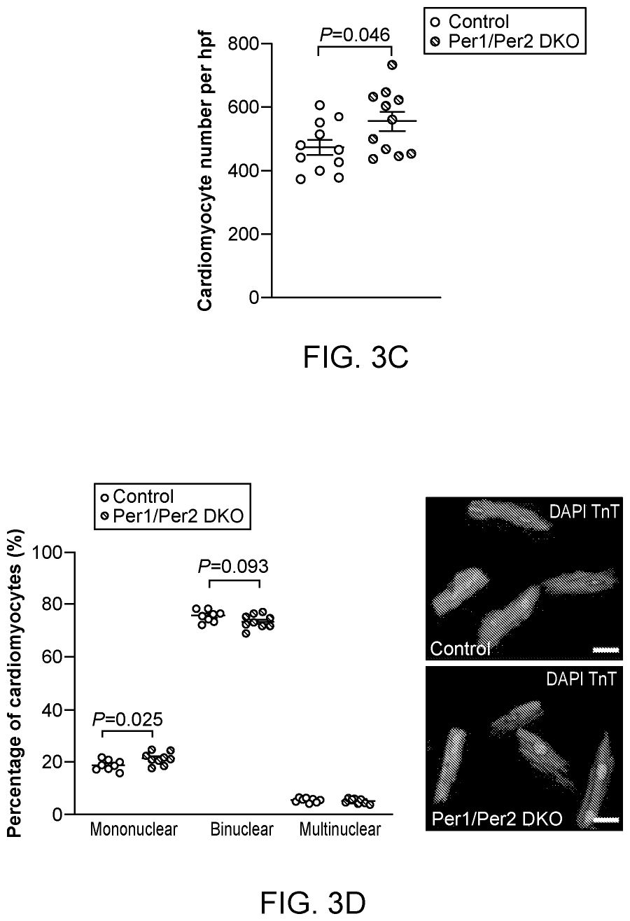

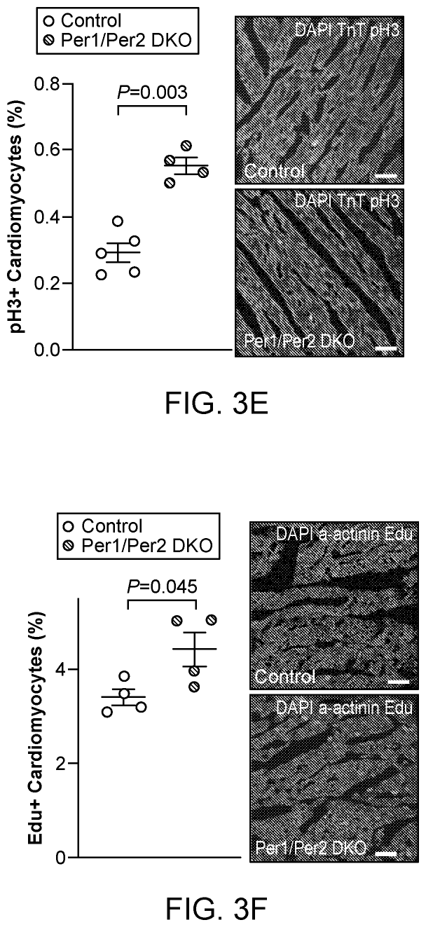

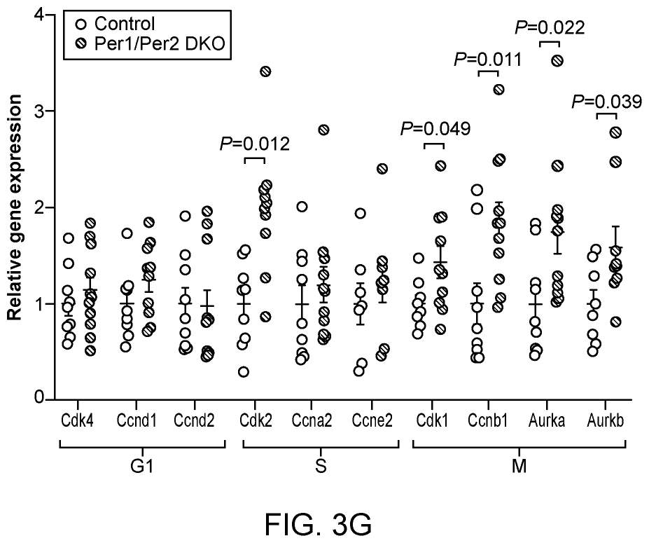

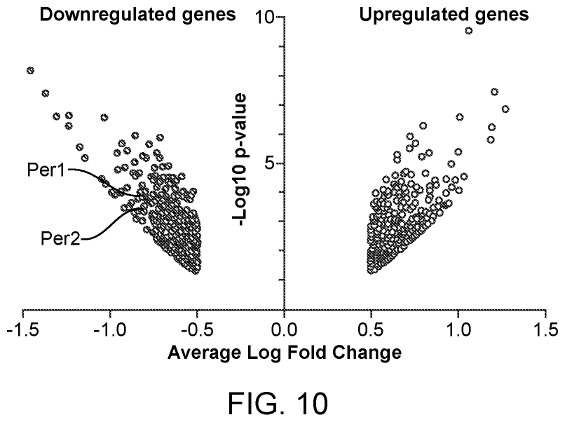

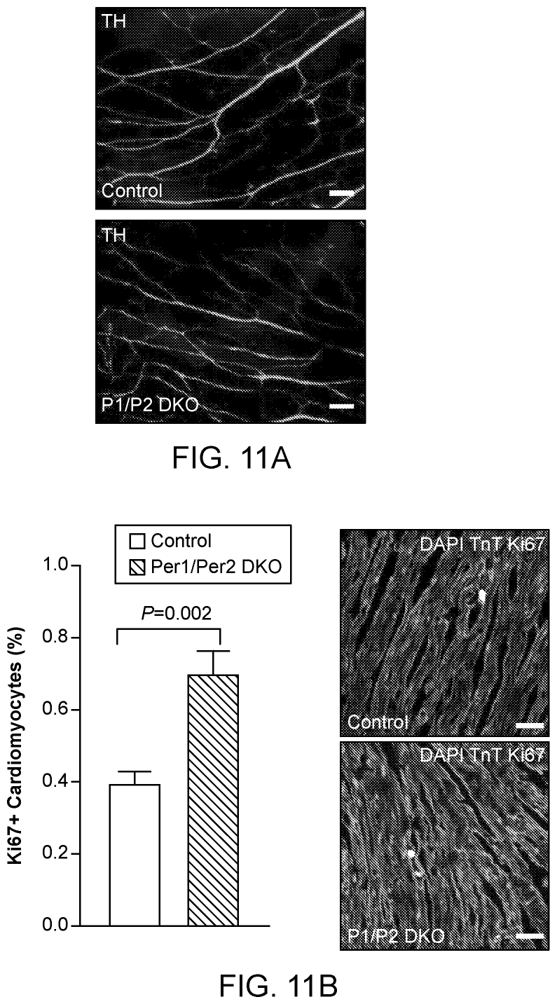

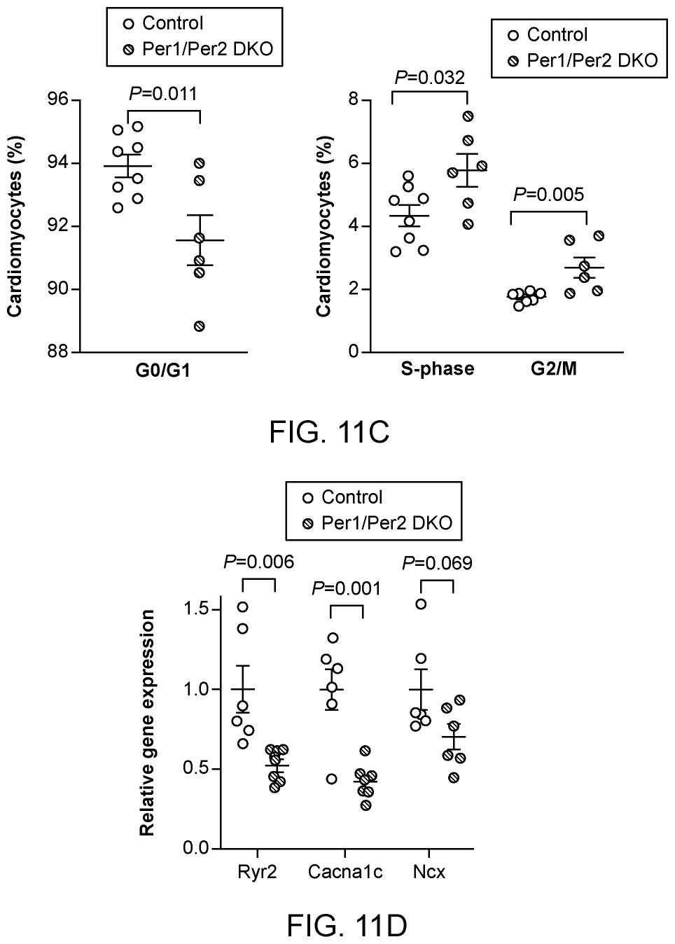

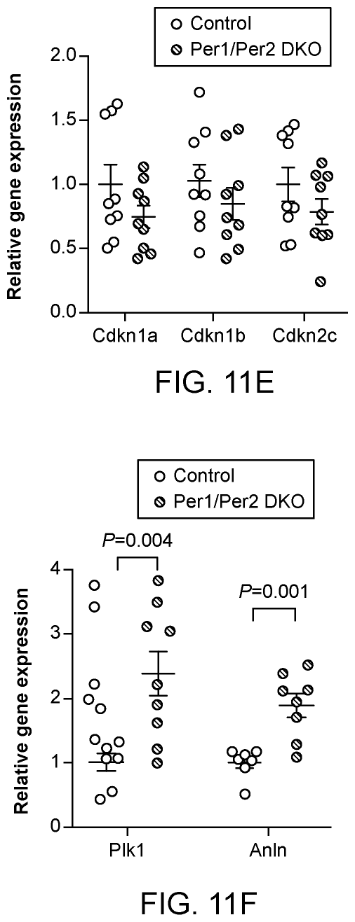

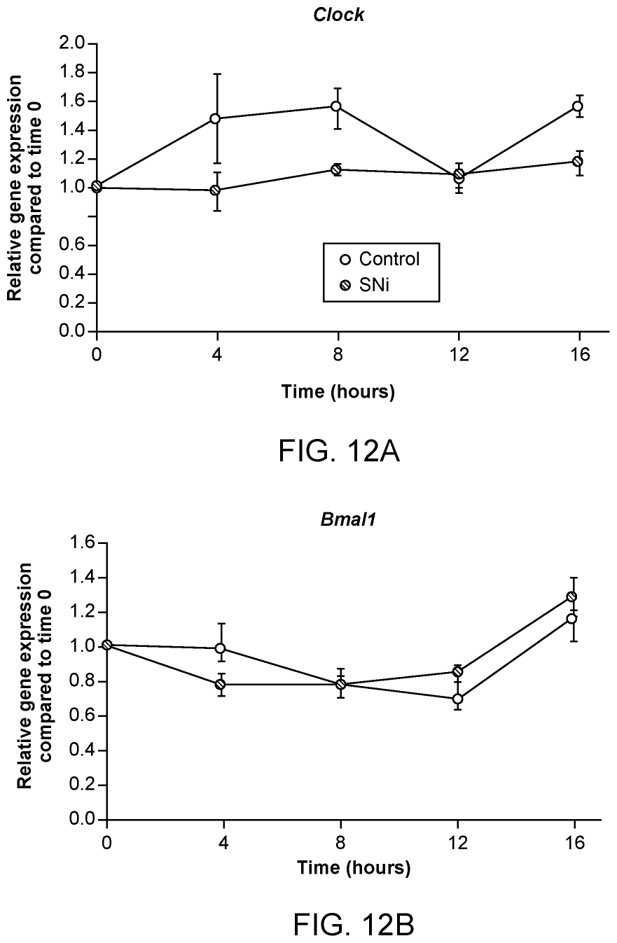

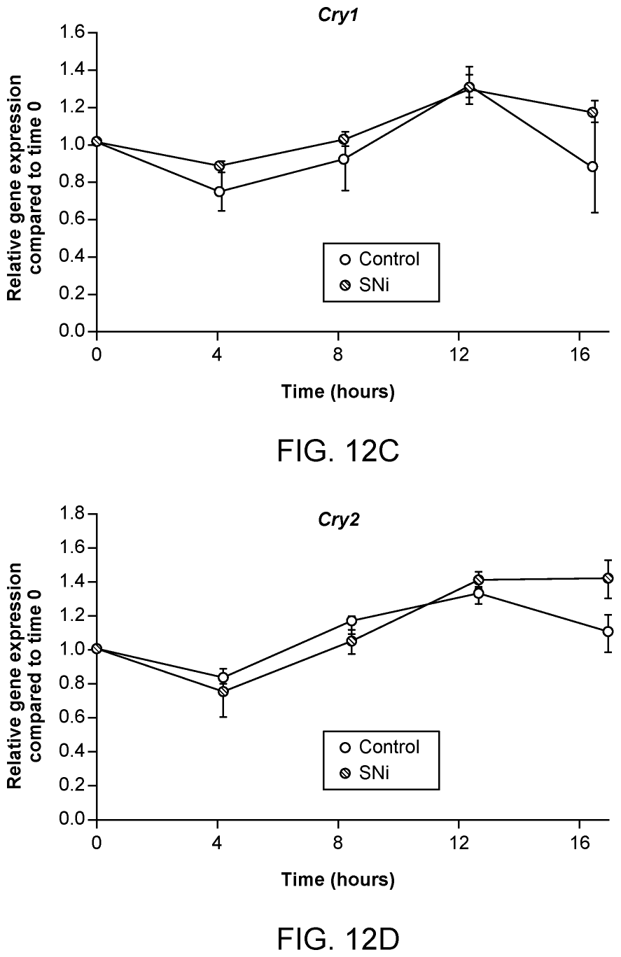

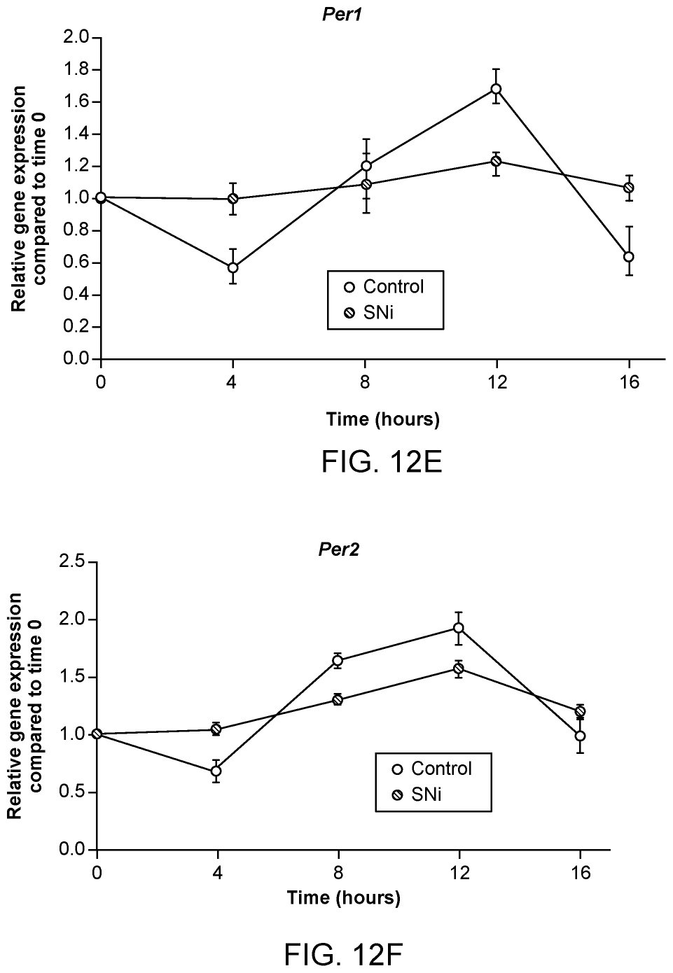

Based on their downregulation in SN-deficient hearts (FIG. 2A), it was tested whether clock genes can affect cardiomyocyte proliferation. To test this, the expression of the main circadian cycle regulators were examined in hearts harvested at random times during the day. Both Period homologs Per1 and Per2 were significantly downregulated in mutant hearts (at P7 and P14), while Bmall expression trended towards lower expression (FIG. 2C, FIG. 10). The decreased Period1 and Period 2 protein expression was verified by western blotting (FIG. 2D). Several calcium handling genes were also found downregulated (FIG. 2E), suggesting SNs affect functional development of cardiomyocytes as well. To examine the specific effect of SNs on circadian gene expression patters, P14 hearts were isolated from SNi and control mice at specific 4-hour intervals, and the expression of the main circadian regulators was analyzed. Per1 and Per2 as well as Clock gene oscillations were suppressed, while the other regulators do not appear to be consistently affected by the lack of sympathetic neurons innervation (FIG. 11). To test whether Per1/Per2 affect heart development, Per1/Per2 double knock-out (DKO) mice were generated. The effect of period proteins on cardiac sympathetic innervation was examined and it was found that cardiac sympathetic innervation was not affected (FIG. 12A). The size of Per1/Per2 DKO mouse hearts was increased at P14 and cardiomyocyte cell size was decreased (FIG. 3A-3B). Subsequent analysis showed that the total number of cardiomyocytes was increased in Per1/Per2 DKO mouse hearts (FIG. 3C), and there was a moderate but significant increase in the percentage of mononuclear cardiomyocytes (FIG. 3D). Moreover, Per1/Per2 DKO resulted in increased percentages of cardiomyocytes positive for Ki67, Edu, and pH3, suggesting more cells are undergoing DNA replication and entering mitosis (FIG. 3E-3F, FIG. 11B). Cell cycle analysis also showed that a higher percentage of Per1/Per DKO cardiomyocytes were in the S and mitosis phase, while a smaller percentage of cells remained in G0/G1 phase (FIG. 12C). This was further supported by increased expression of primarily mitosis-related genes in Per1/Per2 DKO hearts (FIG. 3G). Similar to the hearts with inhibited SNs, calcium handling genes were decreased (FIG. 12D). Additionally, cell cycle inhibitors remained unchanged, and cytokinesis-related genes were upregulated (FIG. 12E-12F). Together, these data suggest that Period genes negatively regulate postnatal cardiomyocyte proliferation.

### Norepinephrine Induces Clock Genes to Suppress Mitosis Entry in Cardiomyocytes

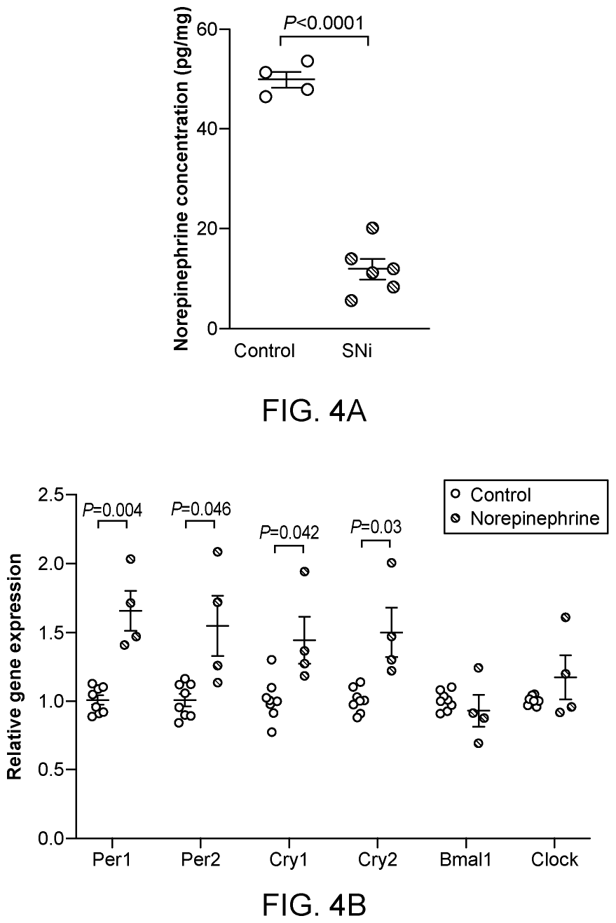

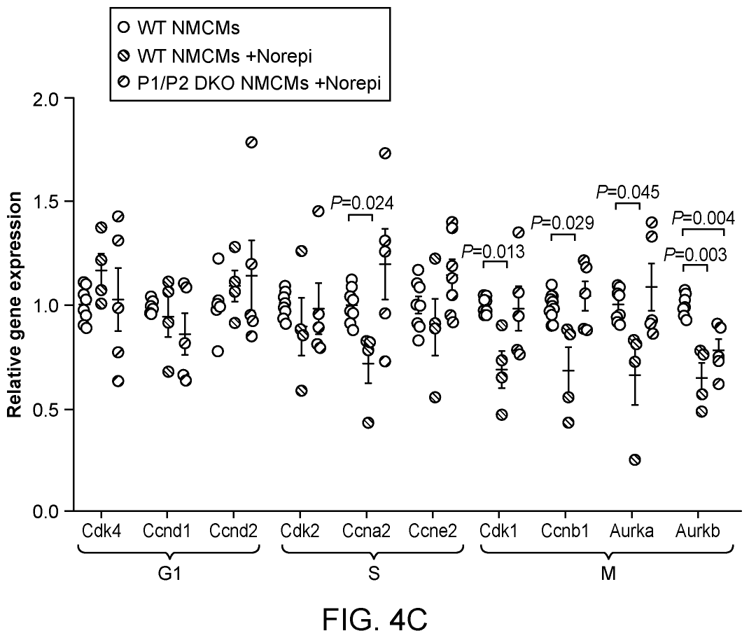

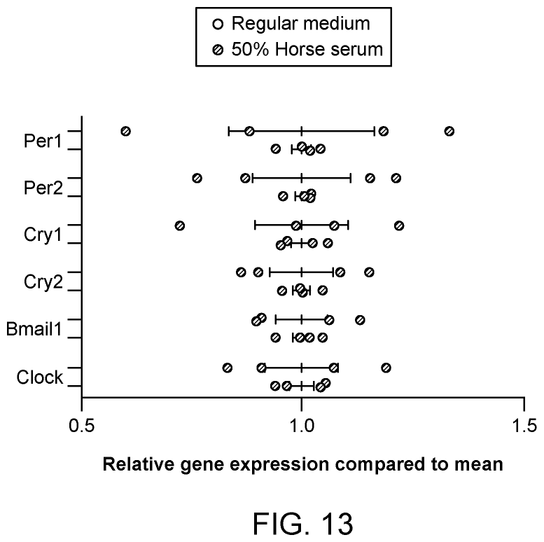

Since norepinephrine is the main post-ganglionic adrenergic neurotransmitter released by SNs, it was examined whether norepinephrine activation of G protein-coupled receptors and increased cAMP levels can induce Per1 and Per2. To test this, the norepinephrine concentration was measured and it was verified that norepinephrine levels were profoundly suppressed in hearts with disrupted SNs, possibly accounting for the reduced Per1 and Per2 expression (FIG. 4A). Next, ex vivo NMCMs were cultured and their cyclic expression was reset by exposing them to 50% serum (FIG. 13). Then the cells were treated with norepinephrine and examined the expression of clock genes after 48 hours. It was found that the treatment induced Per1 and Per2 as well as Cryptochrome1 (Cry1) and Cry2 genes (FIG. 4B). It was further examined whether norepinephrine could decrease cell cycle genes in NMCMs. Consistent with the in vivo data, norepinephrine suppressed mitosis genes (FIG. 4C). However, in the absence of Per1/Per2, there was no significant effect on cell cycle genes apart from Aurkb (FIG. 4C). This suggests that Per1 and Per2 are necessary for the norepinephrine-mediated suppression of mitosis.

### Wee1 Kinase is Suppressed in SN-Deficient and Period1/Period2-Deleted Hearts, Leading to Increased Mitosis Entry

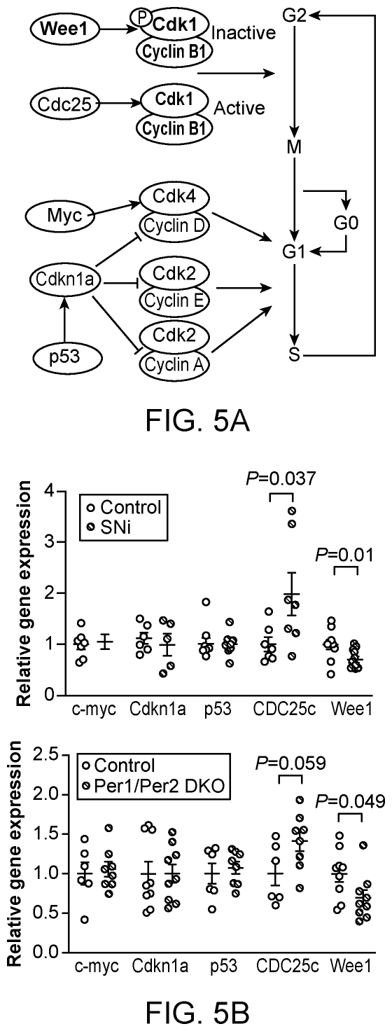

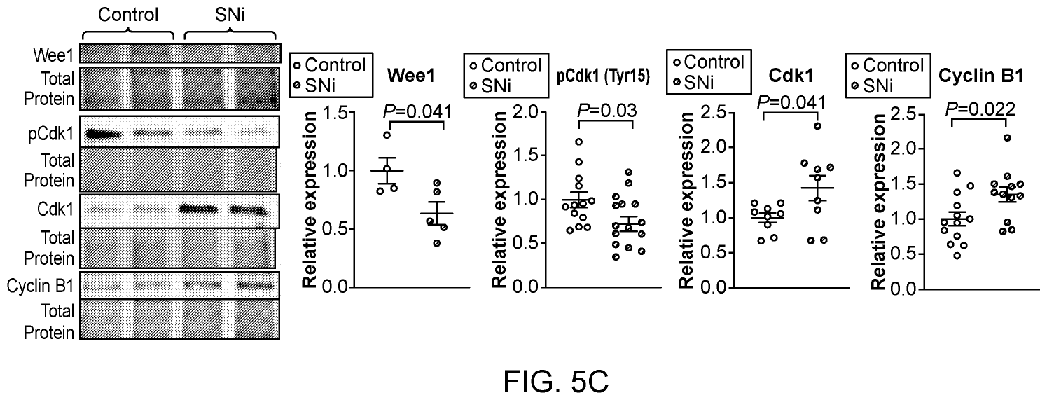

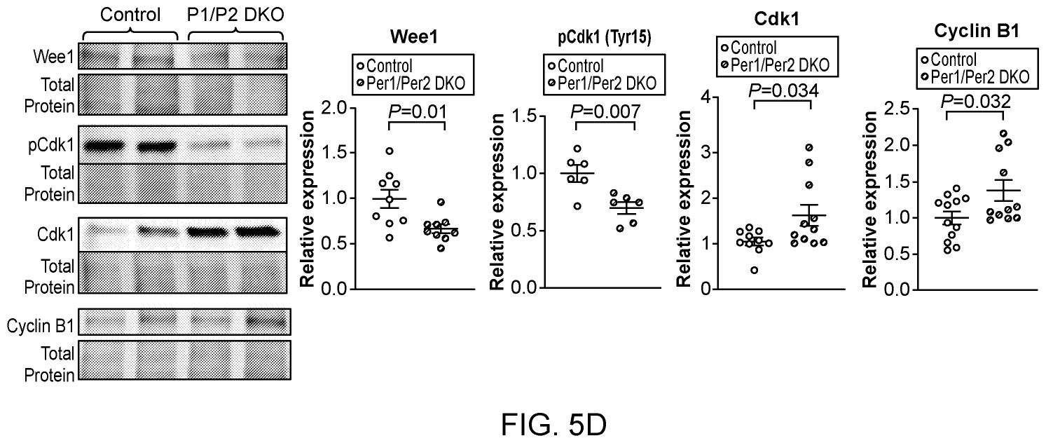

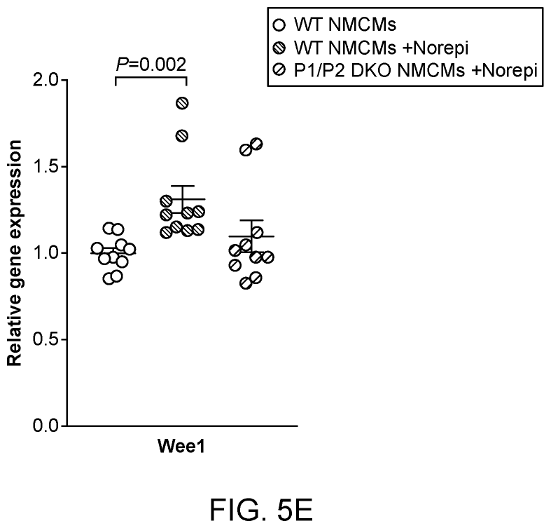

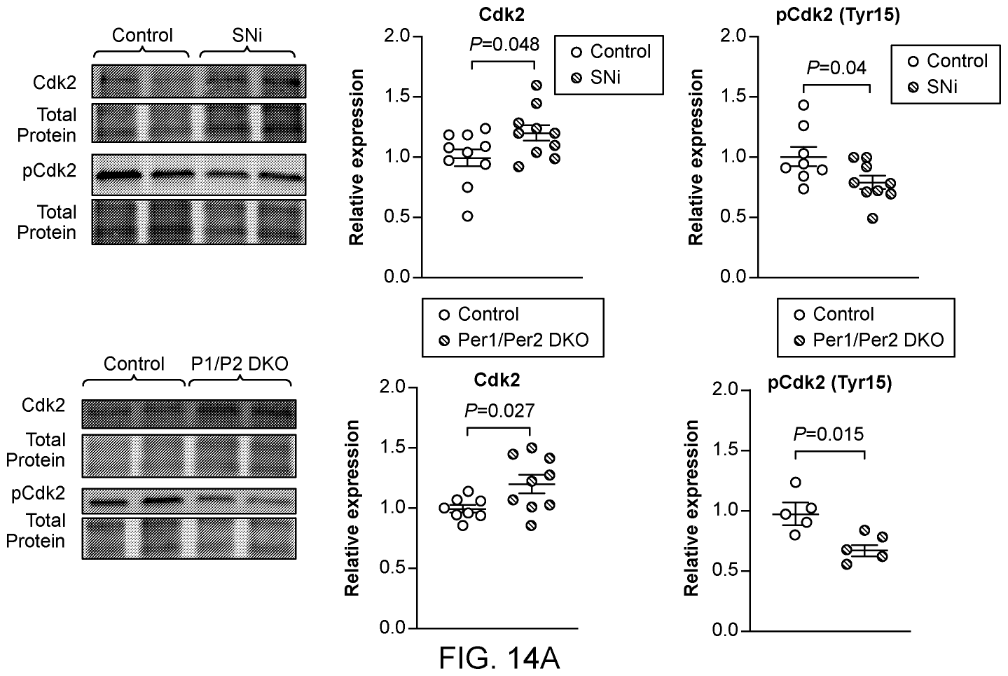

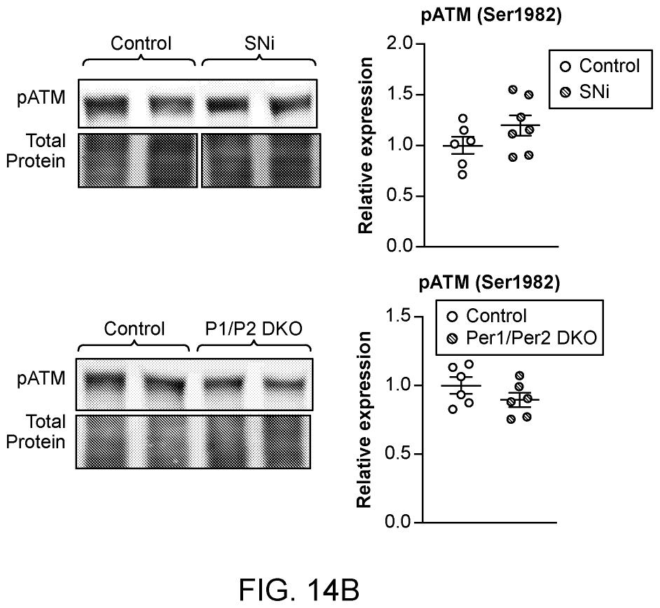

To examine what factors might be regulating cardiomyocyte proliferation and mitosis entry downstream of Per1/Per2, the expression of genes that are known to function as potential coupling factors between circadian and cell cycle factors were analyzed (FIG. 5A). Of all the genes tested, Wee1 (a protein kinase that inactivates Cdk1 by phosphorylation at Tyr15) was consistently downregulated in hearts lacking Per1/Per2 and SNs (FIG. 5B). Contrarily, Cdc25 (a phosphatase with the opposite effect of Wee1) was found increased in SNi hearts (FIG. 5B). It was then verified that Wee1 protein was decreased in hearts lacking SNs or Per1/Per2 (FIG. 5C-5D). This suggests that the clock genes may utilize Wee1 to regulate mitosis entry in cardiomyocytes. To test this, the levels of Cdk1/CyclinB1, a protein complex for mitosis entry whose activity is regulated by Wee1 via phosphorylation, were checked (FIG. 5A). Both mutant hearts showed decreased levels of inactive phospho-Cdk1 (Tyr15) and increased levels of active Cdk1 and Cyclin B1 (FIG. 5C-5D). This suggests that decreased Wee1 expression is associated with higher levels of unphosphorylated and active Cdk1/CyclinB1 complex, resulting in a higher percentage of cardiomyocytes proliferating and entering mitosis. As Wee1 can also phosphorylate Cdk2 at the Tyrosine 15 residue to inhibit cell cycle progression, the regulation of Cdk2 was analyzed. Consistent with the gene expression, Cdk2 protein levels were increased, however the levels of inactive phospho-Cdk2 (Tyr15) remained decreased (FIG. 14A). This suggests that similarly to Cdk1, reduced Wee1 expression can increase the transition to G2/M phase through higher levels of unphosphorylated and active Cdk2. Finally, it was confirmed that norepinephrine induces Wee1 expression in NMCMs, but this effect was not observed in the absence of Per1/Per2 (FIG. 5E). This highlights that Wee1 regulates mitosis entry downstream of norepinephrine induction of Per1 and Per2. No difference in p53 and cell cycle inhibitors were detected in mutant hearts (FIG. 5B). Additionally, there was no difference in levels of phospho-ATM (Ser1981, active form) in either mouse models (FIG. 14B).

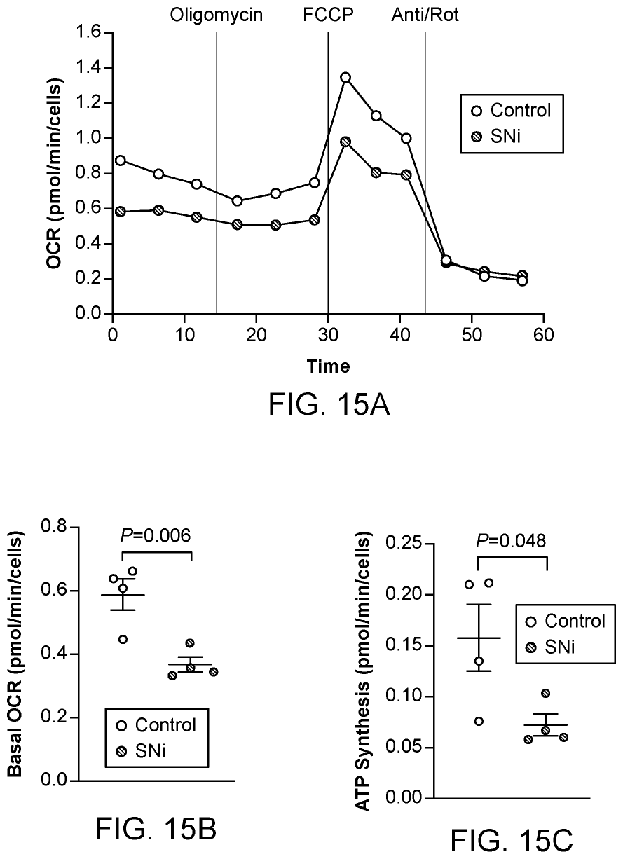

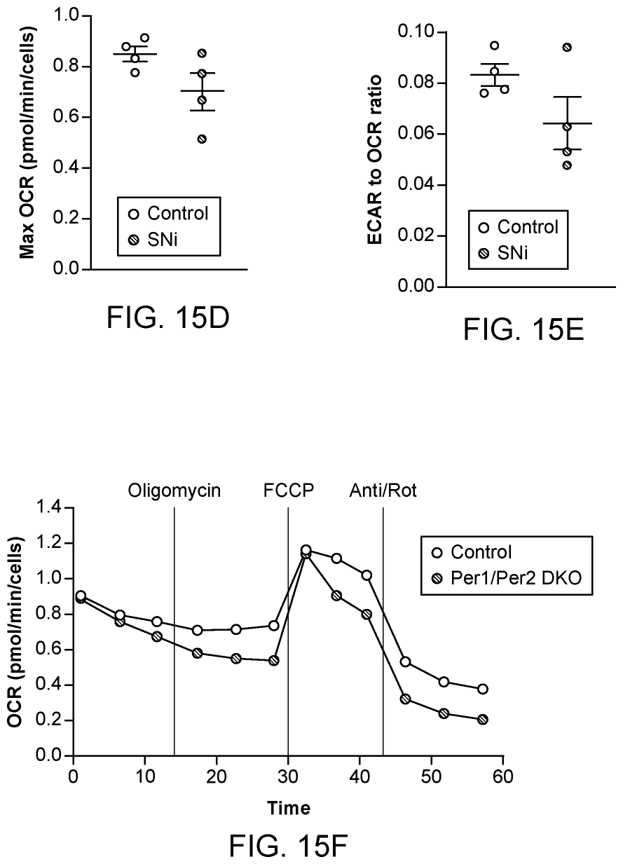

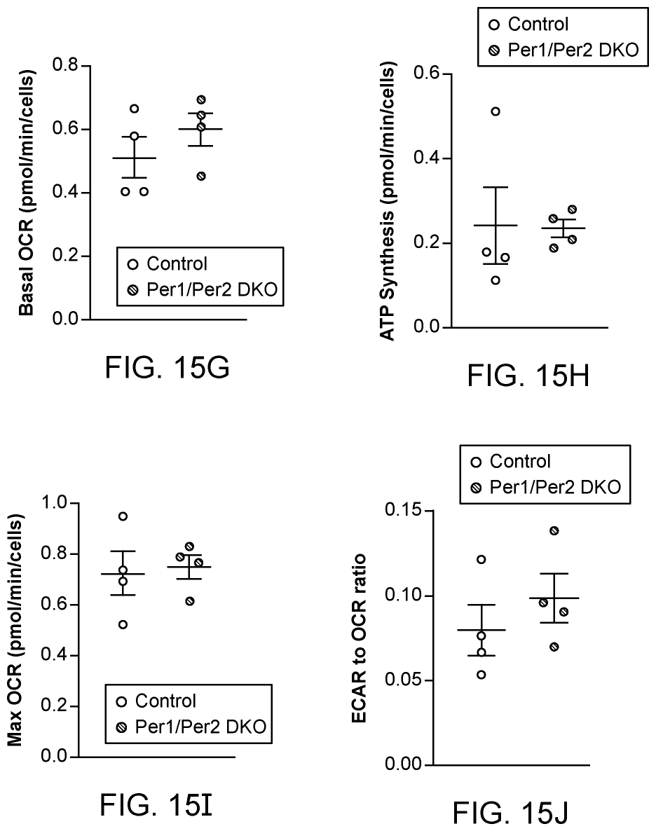

To examine whether suppressed cardiac sympathetic innervation or deletion of Per1/Per2 affects oxygen consumption rate (OCR), Seahorse analysis was performed in isolated postnatal (P14-P18) cardiomyocytes. Basal OCR and ATP synthesis in SNi cardiomyocytes was reduced, while there was no significant difference in glycolysis (ECAR to OCR ratio) (FIG. 15A-15E). In contrast, Per1/Per2 cardiomyocytes did not show significant changes in oxidative metabolism (FIG. 15F-15J). This suggests that in SNi hearts, reduced oxidative metabolism may also influence increased cardiomyocyte proliferation.

### Period2 Binds to Wee1 Promoter

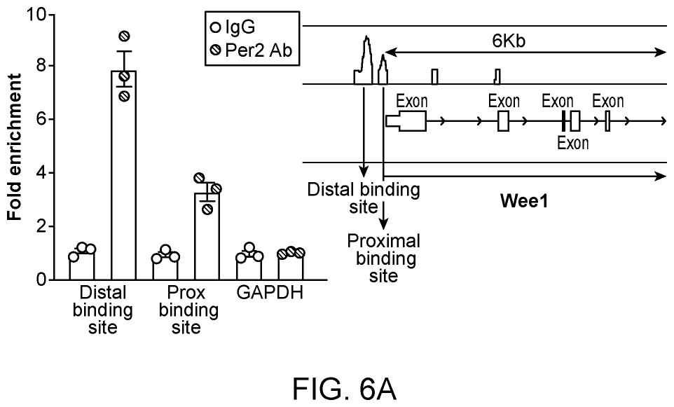

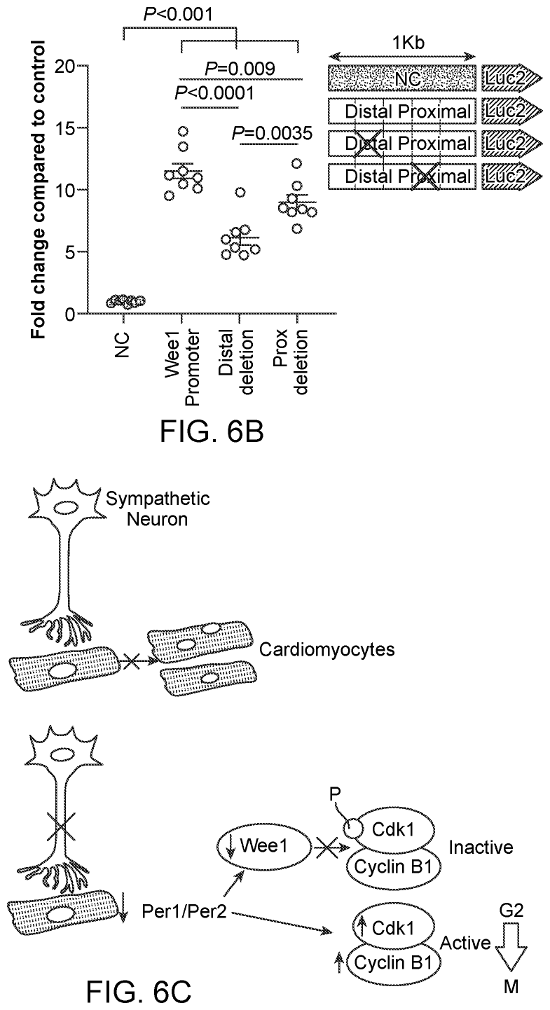

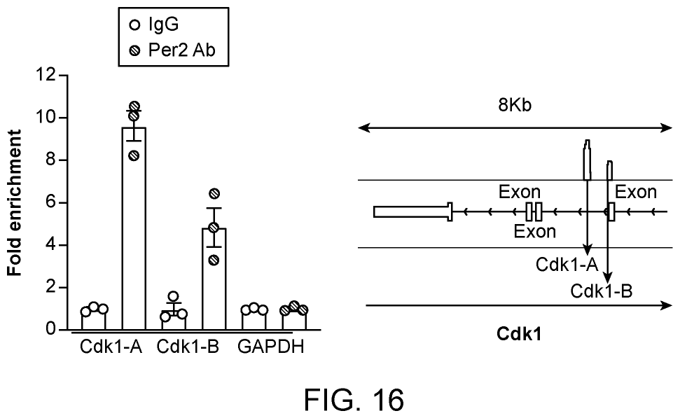

To examine whether Per is physically associated with the Wee1 promoter in postnatal hearts, chromatin immunoprecipitation (ChIP) was performed with Per2 antibody followed by qPCR analysis. Enrichment of two regions within the Wee1 promoter (proximal and distal) bound by Per2 were found, suggesting that Per2 may directly regulate Wee1 expression (FIG. 6A). To test this, luciferase reporter plasmids containing the intact Wee1 promoter with/without the proximal or distal Per2 binding site were constructed and the luciferase activity was analyzed in cardiomyocytes derived from human pluripotent stem cells (PSCs). It was found that both Per2 binding regions can increase luciferase activity with the distal region having a more pronounced effect (FIG. 6B). This is consistent with the ChIP-qPCR data and suggests that these Per2 sites are important for Wee1 expression. It was also found that Per2 can bind a previously described Cdk1 intronic region, suggesting it may regulate Cdk1 expression as well (FIG. 16).

Together these results demonstrate that SNs decrease postnatal cardiomyocyte proliferation. For example, the lack of cardiac SNs can suppress PER1/PER2 polypeptides, and can increase cardiomyocyte proliferation and entry into mitosis (FIG. 6C). Accordingly, one or more inhibitors of a PER polypeptide (e.g., a PER1 polypeptide and/or a PER2 polypeptide) can be used to increase cardiomyocyte proliferation.

### Materials and Methods

### Animals

Sm22a-Cre (017491) and C57BL/6 mice were obtained from Jackson Lab. NGF +/− mice, Per1/Per2 DKO mice, and Mesp1-Cre mice were as described elsewhere (see, e.g., Wheeler et al., Neuron, 82: 587-602 (2014); Muller et al., J. Neurosci., 32: 14885-14898 (2012); and Saga et al., Development, 126: 3437-3447 (1999)). The animals were randomly allocated to experimental groups and both male and female mice were equally used in all experimental assays. All mouse hearts and cardiomyocytes were harvested at random times during the day unless specified otherwise.

### Cardiomyocyte Culture

Cardiomyocytes were isolated from P0 or P1 mouse hearts using a neonatal cardiomyocyte isolation kit (Miltenyi Biotec) based on manufacturer's instructions. Before plating, cardiomyocytes were filtered through a 70 μm mesh and single cells were cultured in 24-well plates coated with gelatin. The cells were first maintained in 5% fetal bovine serum (FBS) in DMEM with Pen/Strep antibiotics for 24 hours and subsequently treated for at least 2 hours with 50% horse serum in DMEM and then for 2 days with 5% FBS in DMEM. Cardiomyocytes were treated with Norepinephrine (1 μM) (Sigma) or Nerve Growth Factor (NGF, 20 ng/ml) (PeproTech) as indicated.

### Luciferase Assay

Human embryonic stem cells (hESC line H9, WiCell Research Institute) were used. hESCs were maintained and differentiated as described elsewhere (Cho et al., Cell Reports, 18: 571-582 (2017)). Briefly, hESCs were maintained in essential 8 medium (ThermoFisher) and they were sequentially treated with 6 μM of CHIR99021 (Tocris, GSK3b inhibitor) for 48 hours followed by 2.5 μM of IWR-1 (Tocris, Wnt signaling antagonist) in RPMI-B27 without insulin (ThermoFisher). Spontaneous beating was noted at day 7 of differentiation. Cardiomyocytes were further selected using sodium lactate (100 mM) for three days. Then cells were replated in gelatin coated plates and 24 hours later they were transfected with the respective vectors using the Lipofectamine Stem reagent (Thermofisher). More specifically, the dual luciferase reporter assay system (Promega) was used and cardiomyocytes were transfected with the modified luciferase vector (pGL4.10, Promega) and the Renilla luciferase vector (pGL4.70, Promega), which was used as internal control. Cardiomyocytes were lysed two days later following the manufacturer's instructions and luciferase levels were measured using the Glomax luminescence plate reader (Promega).

### Whole Mount Immunostaining

Hearts from E18.5 mouse embryos were dissected, fixed in 4% paraformaldehyde and subsequently dehydrated by methanol series and incubated overnight in 20% dimethylsulfoxide/80% methanol solution containing 3% H₂O₂. Hearts were then rehydrated, blocked overnight with 4% BSA in 1% PBS-T and incubated for 48-72 hours with anti-Tyrosine Hydroxylase (TH) antibody (Novus, NB300-109, 1:200), followed by incubation with horseradish peroxidase (HRP)-conjugated antibody (1:500, Abcam). The signal was detected using diaminobenzidine (Sigma). Hearts were refixed and dehydrated by methanol and cleared by benzyl benzoate/benzyl alcohol (2:1). Imaging was performed using a Zeiss stereoscopic microscope. For whole mount immunofluorescence staining, pups were euthanized and fixed in 4% paraformaldehyde for 24 hours. Hearts were dissected and cut in half, blocked with 10% goat serum in PBS-T and incubated overnight with anti-TH or anti-AChT antibodies. Then hearts were stained with Alexa fluor secondary antibody (594) (Life Technologies, 1:500), mounted and imaged using EVOSfl (AMG) microscope.

### Immunohistochemistry

Hearts were fixed in 4% paraformaldehyde, then placed in 30% sucrose followed by OCT and sectioned. For immunofluorescent staining they were blocked for 1 hour with 1% BSA and incubated overnight with the following primary antibodies: α-actinin (Abcam, ab68167), Troponin-T (ThermoFisher, MS-295-P1), phospho-Histone 3 (Millipore, 05-806), Ki67 (Abcam, ab15580), TH (Novus, NB300-109). Alexa fluor secondary antibodies (488, 594, 647) (Abcam, Life Technologies) were used for secondary detection and DAPI was added for nuclei staining. To assess cardiomyocyte size, heart sections were stained with WGA-Alexa fluor 647 (W32466). Cells were imaged using a Leica SP8 confocal microscope. All imaging analysis was performed by two blinded investigators using Image J (Version 1.52q).

### Mouse Electrocardiogra

Electrocardiogram (ECG) recordings were performed using adult mice. Briefly, 6-week old mice were anesthetized with 4% isoflurane, intubated, and placed on ventilator support. The animal's upper back was opened with a small midline incision, and ECG leads were implanted subcutaneously and sutured over the trapezius muscle on both sides. Body temperature was maintained at 37° C. Immediately following implantation, the wound was sutured. ECG was subsequently recorded continuously using the Powerlab data acquisition device and LabChart 8 software (AD instruments). Mice were kept at a stable temperature with regular 12-hour light/dark cycle. ECGs were recorded in conscious animals for approximately 7 days for each mouse. To exclude the effect of increased vagal nerve activation as a result of pain and anesthesia, ECG recordings after day 4 were exclusively analyzed. Heart rate variability analysis was performed using LabChart 8. More specifically, the heart rates were averaged over 12 hours (daytime and nighttime separately) and 6 independent average values per animal (3 days of total recording) were analyzed.

### Heart Dissociation and Flow Cytometry

Harvested hearts were placed in a Langendorff system and perfused with a Type II Collagenase (Worthington) and Protease (Sigma) digestion buffer. Whole hearts were then triturated until no residual undigested tissue and filtered through a mesh. Single cardiomyocytes were subsequently fixed with 4% PFA for one hour and then washed with PBS. For flow cytometry, cells were permeabilized with saponin (Sigma), stained with troponin T (Thermo Scientific, MS-295-P1) and either phospho-Histone 3 (Milipore, 05-806) or Ki67 (Abcam, ab15580), followed by incubation with Alexa fluor secondary antibodies (488, 647) (Abcam). For EdU analysis, the Click-iT EdU kit (Life Technologies) was used followed by immunostaining with primary and secondary antibodies. Cell cycle analysis was performed using cardiomyocytes isolated from P7 mice after staining with Hoechst 33342 (Invitrogen) and cardiac Troponin T. Flow cytometry was performed using BD Accuri C6. Data was analyzed using FlowJo software (version 10.5). To examine cardiomyocyte number and nucleation, cells were similarly stained for Troponin T and DAPI and imaged using EVOSfl (AMG). All samples were analyzed by two blinded investigators.

### RNA-Sequencing

Hearts were dissected, washed in PBS, and homogenized in Trizol (ThermoFisher) and RNA was isolated following the manufacturer's instructions. All hearts for each time point were isolated at the same time (2 pm for P7 and 10 am for P14). cDNA libraries for bulk-RNA sequencing were prepared using the TruSeq kit (Illumina) and sequenced using HiSeq 2500. Raw sequencing reads were trimmed using Trimmomatic (0.36) with a minimum quality threshold of 35 and minimum length of 36. Processed reads were mapped to the mm 10 reference genome using HISAT2 (2.0.4). Counts were then assembled using Subread featureCounts (1.5.2) in a custom bash script. Differential gene expression analysis was done using the DESeq2 package in R. Gene ontology (GO) analysis was performed using PANTHER. Canonical pathway analysis was done using Ingenuity Pathway Analysis (QIAGEN Inc.).

### Quantitative PCR

RNA was isolated from mouse hearts and cultured cardiomyocytes using Trizol, and cDNA was generated using the high-capacity cDNA reverse transcription kit (Applied Biosystems). qPCR reactions were performed using the Sybr Select qPCR mix (Thermo Fisher) with indicated primers. Gene expression levels were normalized to Gapdh or Rpl32.

### Western Blotting

Protein sample preparation from mouse ventricles was performed with tissue homogenization in Cell Lysis buffer (Cell Signaling) with added PMSF (Sigma) and PhosStop (Roche). Protein concentration was determined by bicinchoninic acid assay (Pierce). Electrophoresis was performed using 4% to 20% tris-glycine TGX gels (Bio-Rad) and proteins were transferred onto nitrocellulose membranes. The following primary antibodies were used for immunoblotting: TH (Novus NB300-109, 1:1000), Perl (Biolegend, 936002 1:1000), Per2 (Abcam Ab180655, 1:1000), Wee1 (Abcam Ab137377, 1:1000), phospho-Cdk1 (Y15) (Cell Signaling 9111T, 1:1000), Cdk1 (Novus, NBP-2 67438, 1:1000), Cyclin B1 (Santa Cruz, SC-245, 1:1000), phospho-ATM (S1982) (Santa Cruz, SC-47739, 1:1000), Cdk2 (Cell Signaling, 2546S, 1:500), phospho-Cdk2 (Y15) (Novus, NBP2-67686 1:1000). IRDye secondary-fluorescent conjugated antibodies were used (Li-Cor, 1:20000). Total protein staining was performed for sample normalization (926-11016; Li-Cor). Antibody binding was visualized with an infrared imaging system (Odyssey, Li-Cor) and band quantification performed with Image Studio 5.2.5 (Li-Cor).

### Oxygen Consumption Measurement

Respiration rates were measured with Seahorse XFe96 Analyzer. Cardiomyocytes isolated from P14-P21 mice were plated at ˜2×10⁴ cells per well of a 96 well XF96 Cell Culture Microplate (Aligent Technologies) and cultured for ˜1 hour in Seahorse assay medium (0.55 mg/ml pyruvate in base medium, pH7.4). After determination of basal oxygen consumption rates, cells were treated with oligomycin A (1 μM), FCCP (1 μM), and rotenone (1 μM) with antimycin A (1 μM). Cardiomyocyte numbers were counted and used to normalize the oxygen consumption rate.

### Chromatin Immunoprecipitation (ChIP) Analysis

DNA isolation for ChIP qPCR analysis was performed as described elsewhere (van den Boogaard et al., Meth. Mol. Biol., 977: 53-64 (2013)). Briefly, several hearts (4-5 hearts per sample) were isolated from P7 WT C57BL/6 mice, fixed in 1% paraformaldehyde followed by quenching with glycine. The heart tissue was subsequently homogenized and lysed using 1% SDS lysis buffer and protease inhibitor cocktail (Roche). DNA was fragmented using 20 cycles of 30 seconds “on” then 30 seconds “off” of 50% power sonication and DNA shearing efficiency was confirmed by DNA electrophoresis. Samples were subsequently incubated with protein G magnetic beads for 1 h. 2.5% of samples was kept as input (reference sample). Per2 ChIP-grade antibody (Novus, NB100-125) was added, and samples were incubated at 4° C. overnight. Protein G magnetic beads were incubated for 1 hour then pulled down. Chromatin was eluted from the beads then un-crosslinked by incubating overnight at 65° C. DNA was purified using phenol-chloroform following RNase A and Proteinase K treatment (Thermo Fisher). DNA concentrations were calculated using the DNA Qubit 4 Fluorometer (Thermo Fisher). qPCR was performed using Sybr Select qPCR mix (Thermo Fisher) with specific primers. Fold enrichment of DNA fragments compared to the input samples was calculated.

### Heart Norepinephrine Measurement

P14 mouse hearts were harvested and placed in 0.01 N HCl with EDTA (1 mM) and sodium metabisulfite (4 mM) (pH>7.0) to prevent norepinephrine degradation and hearts were homogenized and stored in −80° C. Norepinephrine concentration was calculated using the Norepinephrine Elisa Kit (Abnova, KA3836) following the manufacturer's instructions.

### Statistical Analyses

All studies in cultured cells were performed using at least 4 sets of independent experiments. For in vivo studies at least 5 animals in each group were analyzed. Student's t-test was used for two group analysis. ANOVA with appropriate corrections for post-hoc analysis was used for multiple group comparisons. p value<0.05 was considered significant. Data were presented as mean±SEM. Graphs and statistical analysis were performed using Graphpad Prism V8. For RNA-seq analysis, Benjamini-Hochberg correction was used to adjust for multiple testing, with threshold of adjusted p-value<0.05 (i.e., false discovery rate<10%) considered significant. For Gene Ontology analysis only pathways with p-value<10⁻⁵ were reported.

### Example 2: Treating a Human Having Heart Disease

A human having, or at risk of having, heart disease (e.g., a heart attack or heart failure) is administered one or more inhibitors of PER polypeptide expression or activity. The administered inhibitor(s) can reduce the severity of one or more symptoms of the heart disease.

### Example 3: Treating a Human Having Heart Disease

A human having, or at risk of having, heart disease (e.g., a heart attack or heart failure) is administered one or more inhibitors of PER polypeptide expression or activity. The administered inhibitor(s) can increase the amount of cardiomyocyte proliferation in the heart of the human.

### Example 4: Treating a Human Having Heart Disease

A human having, or at risk of having, heart disease (e.g., a heart attack or heart failure) is administered one or more inhibitors of PER polypeptide expression or activity. The administered inhibitor(s) can regenerate cardiomyocytes in the heart of the human.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having a heart disease, wherein said method comprises administering an inhibitor of a period circadian protein (PER) polypeptide to said mammal.

2. A method for increasing proliferation of a cardiomyocyte within a heart of a mammal having a heart disease, wherein said method comprises administering an inhibitor of a PER polypeptide to said mammal.

3. A method for regenerating a cardiomyocyte within a heart of a mammal having a heart disease, wherein said method comprises administering an inhibitor of a PER polypeptide to said mammal.

4. A method for increasing a number of cardiomyocytes within a heart of a mammal having a heart disease, wherein said method comprises administering an inhibitor of a PER polypeptide to said mammal.

5. The method of claim 1, wherein said mammal is a human.

6. The method of claim 1, wherein the heart disease is a heart attack or heart failure.

7. The method of claim 1, wherein said inhibitor of said PER polypeptide can inhibit one or more of a PER1 polypeptide, a PER2 polypeptide, and a PER3 polypeptide.

8. The method of claim 1, said method comprising identifying said mammal as having said heart disease prior to said administering.

9. The method of claim 1, wherein said inhibitor of said PER polypeptide is an inhibitor of PER polypeptide activity.

10. The method of claim 1, wherein said inhibitor of said PER polypeptide is an inhibitor of PER polypeptide expression.

11. The method of claim 10, wherein said inhibitor of said PER polypeptide expression is a nucleic acid molecule designed to induce RNA interference of said PER polypeptide expression.

12. The method of claim 11, wherein said nucleic acid molecule designed to induce RNA interference of said PER polypeptide expression is a short interfering RNA (siRNA).

13. The method of claim 11, wherein said nucleic acid molecule designed to induce RNA interference of said PER polypeptide expression is a short hairpin RNA (shRNA).

14. The method of claim 1, said method further comprising administering an agent used to treat a heart attack to said mammal.

15. The method of claim 14, wherein said agent is selected from the group consisting of aspirin, thrombolytics, antiplatelet agents, anti-clotting agents, pain relievers, nitroglycerin, beta blockers, ace inhibitors, and statins.

16. The method of claim 1, said method further comprising subjecting said mammal to a therapy used to treat heart disease.

17. The method of claim 16, wherein said therapy is selected the group consisting of, coronary angioplasty, coronary stenting, and coronary artery bypass surgery.

18-20. (canceled)
